(19) United States
(12) Patent Application Publication
(10) Pub. No.: US 2025/0314672 A1
(43) Pub. Date: Oct. 9, 2025

(54) SYSTEMS, DEVICES, AND METHODS FOR RUMINANT ACIDOSIS DETECTION

(71) Applicant: Native Microbials, Inc., San Diego, CA (US)

(72) Inventor: Mallory EMBREE, San Diego, CA (US)

(21) Appl. No.: 18/864,244

(22) PCT Filed: May 10, 2023
(86) PCT No.: PCT/US2023/066851

§ 371 Date: Nov. 8, 2024

Related U.S. Application Data
(60) Provisional application No. 63/340,146, filed on May 10, 2022.

(51) Int. Cl. G01N 33/98; A01K 29/00; A23K 10/18; A23K 50/10; A61D 99/00; C12Q 1/04; G01N 33/84

(52) U.S. Cl. CPC G01N 33/98; A01K 29/005; A23K 10/18; A23K 50/10; A61D 99/00; C12Q 1/04; G01N 33/84

(57) ABSTRACT

Embodiments include example systems, devices, and methods for solvent analysis and biological sample analysis, and for analyzing biological samples for solvents associated with health status related to ruminant acidosis, and for on-farm identification of acidosis in ruminants. In certain aspects, acidosis is detected via measurement of solvents and other volatile chemicals present in the ruminants. Embodiments include methods and systems of diagnosing a state of health of an animal by receiving a sample of a bodily fluid or gas from an oral or nasal cavity of the animal, measuring a quantity of at least one of an identified solvent or an identified gas in the sample, and determining a state of health associated with the animal based on the quantity of the identified solvent.

### CROSS-REFERENCE

The present application claims the benefit of U.S. Provisional Application No. 63/340,146, filed May 10, 2022, which application is incorporated herein by reference in its entirety.

### STATEMENT REGARDING SEQUENCE LISTING

The sequence listing associated with this application is provided in text format in lieu of a paper copy, and is hereby incorporated by reference into the specification. The name of the text file containing the sequence listing is ASBI_027_01WO_SeqList_ST26.xml. The text file is approximately 17,722,115 bytes, was created on May 10, 2023, and is being submitted electronically.

### TECHNICAL FIELD

The present disclosure relates generally to systems, devices, and methods for solvent analysis and biological sample analysis, and specifically relates to analyzing biological samples for solvents associated with health status related to ruminant acidosis. The present disclosure is directed to on-farm identification of acidosis in ruminants. In certain aspects, acidosis is detected via measurement of solvents and other volatile chemicals present in the ruminants.

### BACKGROUND

Livestock animals in modern farms frequently develop acidosis, a metabolic status that is believed to arise due to overproduction of lactate in the rumen. Traditionally, it is believed that acidosis occurs when ruminants consume excessive amounts of rapidly fermentable carbohydrates and/or low amounts of fiber. The microorganisms residing in the rumen ferment the carbohydrates into lactate and other volatile fatty acids, which in turns lowers the pH of the rumen causing a wide range of pathophysiological consequences in the animal.

The main signs attributed to ruminal acidosis are decreased feed intakes, decreased milk production, poor body condition, diarrhea, and generally lowered animal performance. More serious symptoms include rumenitis, rumen ulcers, and inflammation of the epithelium which can lead to liver abscesses and other serious diseases. Herds with high prevalence of acidosis often experience higher culling rates, increased death, and decreased milk production.

Today, the most common diagnosis of acidosis involves measuring the pH of the rumen. This process typically includes: (1) tubing an animal to extract rumen content or (2) ruminocentisis, both of which are highly invasive and difficult to perform routinely. Subacute acidosis is defined as a ruminal pH between 5.2 and 5.6, while acute acidosis is defined as ruminal pH less than 5.5. To elaborate further, the current industry practice involves (1) putting tubing down a cow's throat, which can accidently kill the cow if the tube gets into the lungs, and then utilization of a motor to suck up rumen content. The industry standard cannot be used frequently to the same cow without severe damage to the animal's throat. If not using the aforementioned methodology of placing tubing down the cow's throat, then the industry resorts to (2) ruminocentisis—i.e. use of a syringe and a long needle to stab into the rumen from the cow's side behind the ribs. One can only do this a few times to the same cow—it is highly invasive and animal welfare becomes an issue. In some implementations, the animal swallows a device that can provide real time pH information. Such devices, however, are not a long term solution, as they are damaged easily and provide inaccurate measurements (e.g., with a drift in the baseline readings). Such methods of using a swallowed device can also only be used in one animal at a time, and performing at scale is generally financially infeasible.

There is currently no reliable, on-farm method to diagnose acidosis in ruminants. Even when pH is measured (i.e., using tubing or ruminocentisis, both of which are very labor intensive), it is often unreliable as acidotic animals will sometime have rumen p>6 depending on the time of day, feeding pattern, etc. Additionally, acidosis can be present even when pH readings are not indicative. That is, animals can suffer from or show symptoms of acidosis without having a suppressed pH (suggesting that pH may not be an effective indicator of an animal having an acidosis). Subacute ruminal acidosis is a major concern in the dairy and beef industries. Losses from production alone were estimated to be $1.12/day/cow in a herd diagnosed with subacute ruminal acidosis.

Thus, there is a need in the art for alternative methods of detecting acidosis in ruminants, and/or ameliorating or treating acidosis, which does not involve the invasive and harmful procedures currently practiced by the industry.

### SUMMARY OF DISCLOSURE

Systems, devices, and methods described herein relate to analysis of biological fluids from oral cavity or nasal cavity of an animal. In some embodiments, systems, devices, and methods described herein relate to detection of one or more solvents in a sample of biological fluids obtained from oral cavity or nasal cavity of an animal.

Embodiments disclosed include a method of diagnosing a state of health of an animal, The method comprises receiving a sample of a bodily fluid or gas from an oral or nasal cavity of the animal. The method further comprises measuring a quantity of at least one of an identified solvent or an identified gas in the sample. The method further comprises determining a state of health associated with the animal based on the quantity of the identified solvent.

The present disclosure relates to methods of detecting acidosis in ruminants. The present methods are non-invasive. Further, the presently taught methods and kits are fast, reliable, and are sensitive to the animal's welfare. The present disclosure includes methods of treating or ameliorating a state of acidosis in a ruminant using additives, including one or more synthetic microbial ensembles, adsorptive ingredients, microbial growth inhibitors, carriers, and/or the like. In some implementations. the one or more additives can be directed to increase or improve a yield or property of a bio product (e.g., milk) from the animal.

The present disclosure provides an orally deliverable composition for increasing milk production or improving milk compositional characteristics in a ruminant, comprising: (a) one or more bacteria comprising a 16S nucleic acid sequence with at least about 97% sequence identity to any one of SEQ ID NOs: 2125-4945; and (b) a carrier suitable for ruminant administration. In some embodiments, the one or more bacteria comprises a 16S nucleic acid sequence selected from the group consisting of SEQ ID NOs: 2125-4945.

In some embodiments, the composition comprises: (a) one or more bacteria comprising a 16S nucleic acid sequence with at least about 97% sequence identity to any one of SEQ ID NOs: 1-30, 2045-2103, 2108, 4104, 4116, 4364, 4620, 4814, 4828, 4921, 4943, 4944, and 4945; and/or (b) one or more fungi comprising an ITS nucleic acid sequence with at least about 97% sequence identity to any one of SEQ ID NOs: 31-60 and 2104-2107.

In some embodiments, the composition comprises: (a) one or more bacteria comprising a 16S nucleic acid sequence selected from the group consisting of SEQ ID NOs: 1-30, 2045-2103, 2108, 4104, 4116, 4364, 4620, 4814, 4828, 4921, 4943, 4944, and 4945; and/or (b) one or more fungi comprising an ITS nucleic acid sequence selected from the group consisting of SEQ ID NOs: 31-60 and 2104-2107.

In some embodiments, the ruminant is a cow. In some embodiments, the ruminant is a calf.

In some embodiments, the ruminant administered the composition exhibits an increase in milk production that leads to an increase in milk yield or an increase in energy-corrected milk.

In some embodiments, the ruminant administered the composition exhibits an improved milk compositional characteristic selected from the group consisting of: an increase in milk fat(s), an increase in milk protein(s), an increase of carbohydrates in milk, an increase of vitamins in milk, an increase of minerals in milk, or combinations thereof.

In some embodiments, the ruminant administered the composition exhibits at least one improved phenotypic trait, selected from the group consisting of: an improved efficiency in feed utilization, improved digestibility, an increase in polysaccharide and lignin degradation, an increase in fatty acid concentration in the rumen, pH balance in the rumen, a reduction in methane emissions, a reduction in manure production, improved dry matter intake, an improved efficiency of nitrogen utilization, or combinations thereof.

In some embodiments, the composition is formulated to protect the one or more bacteria from external stressors prior to entering the gastrointestinal tract of the ruminant. In some embodiments, the composition is formulated to protect the one or more bacteria from oxidative stress. In some embodiments, the composition is formulated to protect the one or more bacteria from moisture.

In some embodiments, the composition is dry. In some embodiments, the composition is combined with food. In some embodiments, the composition is combined with cereal, starch, oilseed cake, or vegetable waste. In some embodiments, the composition is combined with hay, haylage, silage, livestock feed, forage, fodder, beans, grains, micro-ingredients, fermentation compositions, mixed ration, total mixed ration, or a mixture thereof. In some embodiments, the composition is formulated as a solid, liquid, or mixture thereof. In some embodiments, the composition is formulated as a pellet, capsule, granulate, or powder. In some embodiments, the composition is combined with water, medicine, vaccine, vitamin, mineral, amino acid, enzyme, or a mixture thereof.

In some embodiments, the composition is encapsulated. In some embodiments, the composition is encapsulated in a polymer or carbohydrate.

In some embodiments, the one or more bacteria are present in the composition in an amount of at least 102 cells.

In some embodiments, the present disclosure provides a method for increasing milk production or improving milk compositional characteristics in a ruminant, the method comprising orally administering to a ruminant an effective amount of any one of the compositions described herein.

In some embodiments, the ruminant administered the effective amount of the composition exhibits an increase in milk production that leads to a measured increase in milk yield.

In some embodiments, the ruminant administered the effective amount of the composition exhibits an increase in milk production and improved milk compositional characteristics that leads to a measured increase in energy-corrected milk.

In some embodiments, the ruminant administered the effective amount of the composition exhibits an improved milk compositional characteristic selected from the group consisting of: an increase in milk fat(s), an increase in milk protein(s), an increase of carbohydrates in milk, an increase of vitamins in milk, an increase of minerals in milk, or combinations thereof.

In some embodiments, the ruminant administered the effective amount of the composition exhibits at least a 1% increase in the average production of: milk fat(s), milk protein(s), energy-corrected milk, or combinations thereof.

In some embodiments, the ruminant administered the effective amount of the composition exhibits at least a 10% increase in the average production of: milk fat(s), milk protein(s), energy-corrected milk, or combinations thereof.

In some embodiments, the ruminant administered the effective amount of the composition exhibits at least a 20% increase in the average production of: milk fat(s), milk protein(s), energy-corrected milk, or combinations thereof.

In some embodiments, the ruminant administered the effective amount of the composition, further exhibits at least one improved phenotypic trait, selected from the group consisting of: an improved efficiency in feed utilization, improved digestibility, an increase in polysaccharide and lignin degradation, an increase in fatty acid concentration in the rumen, pH balance in the rumen, a reduction in methane emissions, a reduction in manure production, improved dry matter intake, an improved efficiency of nitrogen utilization, or combinations thereof.

In some embodiments, the ruminant administered the effective amount of the composition, further exhibits a shift in the microbiome of the rumen.

In some embodiments, the ruminant administered the effective amount of the composition, further exhibits a shift in the microbiome of the rumen, wherein a population of microbes present in the rumen before administration of the composition increase in abundance after administration of the composition.

In some embodiments, the ruminant administered the effective amount of the composition, further exhibits: a shift in the microbiome of the rumen, wherein a population of microbes present in the rumen before administration of the composition decrease in abundance after administration of the composition.

In some embodiments, the ruminant administered the effective amount of the composition, further exhibits: a shift in the microbiome of the rumen, wherein a first population of microbes present in the rumen before administration of the composition increase in abundance after administration of the composition, and wherein a second population of microbes present in the rumen before administration of the composition decrease in abundance after administration of the composition.

In some embodiments, the present disclosure provides a composition that performs the same or better than recombinant bovine growth hormone for increasing milk production or improving milk compositional characteristics in a ruminant, wherein the composition comprises: (a) one or more bacteria comprising a 16S nucleic acid sequence with at least about 97% sequence identity to any one of SEQ ID NOs: 2125-4945; and (b) a carrier suitable for ruminant administration.

In some embodiments, the one or more bacteria comprises a 16S nucleic acid sequence selected from the group consisting of SEQ ID NOs: 2125-4945.

In some embodiments, the composition comprises: (a) one or more bacteria comprising a 16S nucleic acid sequence with at least about 97% sequence identity to any one of SEQ ID NOs: 1-30, 2045-2103, 2108, 4104, 4116, 4364, 4620, 4814, 4828, 4921, 4943, 4944, and 4945; and/or (b) one or more fungi comprising an ITS nucleic acid sequence with at least about 97% sequence identity to any one of SEQ ID NOs: 31-60 and 2104-2107.

In some embodiments, the composition comprises: (a) one or more bacteria comprising a 16S nucleic acid sequence selected from the group consisting of SEQ ID NOs: 1-30, 2045-2103, 2108, 4104, 4116, 4364, 4620, 4814, 4828, 4921, 4943, 4944, and 4945; and/or (b) one or more fungi comprising an ITS nucleic acid sequence selected from the group consisting of SEQ ID NOs: 31-60 and 2104-2107.

Rapid detection of this disease state will enable dairy owners and nutritionists to take the appropriate steps to resolve and quickly enact operational changes to improve the health and productivity of their herd (i.e. through diet changes).

Furthermore, recent analyses suggest that acidosis arises due to an increase in the microbial biomass (cells/mL) of gastrointestinal content. An increased number of cells leads to increased concentrations of not only acids, but carbon dioxide, hydrogen, solvents, and other microbial by products. Accumulation of carbon dioxide, in particular, can reduce the buffering ability of bicarbonate in the rumen allowing pH to decline more rapidly and severely. Described herein is a new underlying causal mechanism associated with acidosis that is unexpected and counter to current understanding in industry and academia. Based on the new causal mechanism, embodiments described herein include devices, systems, and methods to detect and/or measure a state of acidosis in ruminants using non-invasive sampling. Also described herein are system and methods to treat, and utilize the information gained from understanding this process.

### BRIEF DESCRIPTION OF THE DRAWINGS

FIG. 1 is a schematic representing changes associated with a progression of acidosis in the rumen of an animal.

FIG. 2 is a schematic illustrating an acidosis detection system (ADS), according to some embodiments.

FIG. 3 is a schematic illustrating a sample examining device included in an ADS, according to some embodiments.

FIG. 4 is a schematic illustrating a compute device included in an ADS, according to some embodiments.

FIG. 5 is a schematic illustrating a progression of acidosis in ruminants.

FIG. 6 is a flow diagram illustrating an example method of acidosis detection using an ADS, according to some embodiments.

FIG. 7 is a flow diagram illustrating an example method of acidosis detection using an ADS, according to some embodiments.

FIG. 8 is an illustration of an example timeline to study effects associated with acidosis and diet in animals using an ADS, according to some embodiments.

FIGS. 9A and 9B show plots of dissolved CO2 as a function of rumen pH measured in the rumen and dissolved carbon dioxide associated with a state and progression of acidosis in an animal.

FIG. 10 is a schematic illustration of progression of acidosis in rumen of ruminants.

FIG. 11A shows a table indicating constitutes in a diet of cow used in Experiment I. FIG. 11B. is a plot of baseline measurements of concentration of ethanol in saliva of healthy cows under feed associated with diet shown in FIG. 11A.

FIGS. 12A is a schematic representation of an example schedule according to an example protocol to study effects of acidosis induction and recovery in an example cohort of animals, using an ADS, according to some embodiments.

FIG. 12B a representation of an example composition of a feed provided to study acidosis induction and recovery in an example cohort of animals, using an ADS, according to some embodiments.

FIGS. 13A, 13B, and 13C are plots of changes in pH in a cohort of animals that were subjected to acidosis challenge, measured using an ADS, according to some embodiments.

FIGS. 14A, 14B, and 14C are plots of changes in rumen biomass in a cohort of animals that were subjected to acidosis challenge, measured using an ADS, according to some embodiments.

FIGS. 15A, 15B, and 15C are plots of intake and concentration of ethanol in a cohort of animals that were subjected to acidosis challenge, measured using an ADS, according to some embodiments.

FIGS. 16A, 16B, and 16C are plots of concentration of ethanol in a cohort of animals that were subjected to acidosis challenge, measured using an ADS, according to some embodiments.

FIGS. 17A, 17B, and 17C are plots of milk yield, fat content, and protein content in milk, respectively, measured in a cohort of animals that were subjected to acidosis challenge, using an ADS, according to some embodiments.

FIG. 18 is a plot of rumen pH as a function of concentration of solvent measured in an experimental cohort of dairy cows, using an ADS, according to an embodiment.

FIG. 19A and 19B are plots of traces indicating a concentration for ethanol obtained from a generated control sample and a sample containing a carrier configured to absorb ethanol, respectively.

FIG. 20 is a plot showing the relative absorption of various carriers tested to identify suitable carriers that can be added as feed supplement for recovery from acidosis, recommended using an ADS, according to an embodiment.

### DETAILED DESCRIPTION

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein the terms “microorganism” or “microbe” should be taken broadly. These terms are used interchangeably and include, but are not limited to, the two prokaryotic domains, Bacteria and Archaea, eukaryotic fungi and protists, as well as viruses. As used herein the terms “microorganism” or “microbe” should be taken broadly. These terms are used interchangeably and include, but are not limited to, the two prokaryotic domains, Bacteria and Archaea, eukaryotic fungi and protists, as well as viruses. In some embodiments, the disclosure refers to the “microbes” of Tables 1, 2, 3, 4, 5, and/or 6, or the “microbes” incorporated by reference. This characterization can refer to not only the predicted taxonomic microbial identifiers of the table, but also the identified strains of the microbes listed in the table.

As used herein, “microbial composition” refers to a composition comprising one or more microbes of the present disclosure, wherein a microbial composition, in some embodiments, is administered to animals of the present disclosure.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); E. W. Martin (1970. Remington's Pharmaceutical Sciences. 17th Ed. Mack Pub. Co.); and Blaser et al. (US Publication US20110280840A1).

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970)(discussing purified prostaglandins), see also, In re Bergy, 596 F.2d 952 (CCPA 1979) (discussing purified microbes), see also, Parke-Davis & Co. v. H.K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

As used herein, “microbiome” refers to the collection of microorganisms that inhabit the digestive tract or gastrointestinal tract of an animal (including the rumen if said animal is a ruminant) and the microorgansims' physical environment (i.e. the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the microbiome of a rumen that can be achieved via administration of the compositions of the disclosure, can take the form of: (a) increasing or decreasing a particular Family, Genus, Species, or functional grouping of microbe (i.e. alteration of the biotic component of the rumen microbiome) and/or (b) increasing or decreasing volatile fatty acids in the rumen, increasing or decreasing rumen pH, increasing or decreasing any other physical parameter important for rumen health (i.e. alteration of the abiotic component of the rumen mircrobiome).

As used herein, “probiotic” refers to a substantially pure microbe (i.e., a single isolate) or a mixture of desired microbes, and may also include any additional components that can be administered to a mammal for restoring microbiota. Probiotics or microbial inoculant compositions of the invention may be administered with an agent to allow the microbes to survive the environment of the gastrointestinal tract, i.e., to resist low pH and to grow in the gastrointestinal environment. In some embodiments, the present compositions (e.g., microbial compositions) are probiotics in some aspects.

As used herein, “prebiotic” refers to an agent that increases the number and/or activity of one or more desired microbes. Non-limiting examples of prebiotics that may be useful in the methods of the present disclosure include fructooligosaccharides (e.g., oligofructose, inulin, inulin-type fructans), galactooligosaccharides, amino acids, alcohols, and mixtures thereof. See Ramirez-Farias et al. (2008. Br. J. Nutr. 4:1-10) and Pool-Zobel and Sauer (2007. J. Nutr. 137:2580-2584 and supplemental).

As used herein, “improved” should be taken broadly to encompass improvement of a characteristic of interest, as compared to a control group, or as compared to a known average quantity associated with the characteristic in question. For example, “improved” milk production associated with application of a beneficial microbe, or consortia, of the disclosure can be demonstrated by comparing the milk produced by an ungulate treated by the microbes taught herein to the milk of an ungulate not treated. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, “inhibiting and suppressing” and like terms should not be construed to require complete inhibition or suppression, although this may be desired in some embodiments.

### 1. System and Devices

Systems, devices, and methods described herein relate to processing of samples of biological bodily fluids or gases from an oral or nasal cavity of an animal to determine a state of health associated with the animal. Embodiments disclosed relate to methods of detecting acidosis in ruminants by measuring solvents (or gas) associated with acidosis in the saliva or secretions in nasal/oral cavity of rumen. The present methods are non-invasive. Further, the presently taught methods and kits are fast, reliable, and are sensitive to the animal's welfare.

While certain examples presented herein may generally relate to processing the sample of bodily fluid or gas to measure a quantity of an identified solvent, ethanol associated with acidosis, it can be appreciated by one of ordinary skill in the art that such systems, devices, and methods can be used to process samples of bodily fluid or gas for any suitable component that is associate with any suitable pathological state, including, for example, components that are by products of processing food in the rumen of ruminants and/or associated with pathologies like laminitis, ketosis, ruminitis, etc.

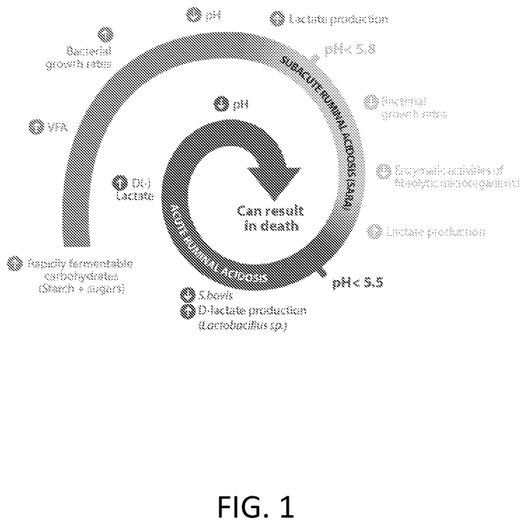

FIG. 1 shows an illustration of a theory of progression of rumen acidosis in a ruminant. The theory of acidosis, as represented in FIG. 1 (adapted from Nocek J. E. 1997) shows how farming practices can be linked to rumen acidosis in animals. High levels of fermentable carbohydrates used in intensive farming practices can challenge rumen stability and increase the risk of ruminants developing metabolic disorders like sub-acute acidosis. Conventional wisdom has attributed lactate accumulation to rumen acidosis. It is considered to progress as shown in FIG. 1, where each successive stage or state of acidosis is associated with ac increased degree of acidosis. Each state of acidosis progresses to the next when the fermentation profile of the rumen is unbalanced by high levels of lactic acid or butyric acid.

The industry and academic field of study associated with rumen health understands that rapid fermentation and acid production leads to decrease in rumen pH and microbiome dysbiosis. As shown in FIG. 1, consumption of rapidly fermentable carbohydrates leads to increased volatile fatty acids (VFAs) and increased bacterial growth rates in the rumen leading to decrease in rumen pH. Many lactic acid producing bacteria are more acid tolerant than other rumen bacteria (Russell, 1992). Therefore, lactic acid producers continue to grow at a low pH resulting in increased lactate production. When a state of subacute ruminal acidosis sets in (when pH decreases below 5.8) bacterial growth rates were thought to drop and enzymatic activities of fibrolytic microbes were thought to decrease whereas lactate production continues to increase further resulting in a state of acute ruminal acidosis which can lead to fatal consequences if left unchecked.

This theory presents gaps that remain for a better understanding of the mechanism underlying ruminal acidosis. For example, the state of ruminal acidosis is not particularly related to pH changes from acid accumulation alone since studies show that acidosis cannot be induced through acid infusion into the rumen (i.e. drive pH decline). Neither can acidosis be induced through lactate infusion into the rumen (i.e. increase lactate concentration). Studies indicate that acidosis can only be reliably induced through diet, via an increase in concentrate and a decrease in fiber, and/or increase in polyunsaturated fatty acids (PUFAs). This leads to the modified understanding that consumption of the increased concentrate/decreased fiber leads to increased biomass and release of carbon dioxide in a first state of acidosis, which then leads to the second state of acidosis. This second state of acidosis is first marked by increased production of fermentation acids and a decline in pH, which is followed by a shift in microbial metabolism towards fermentation solvents produced by the increased biomass. The second state then leads to pathophysiologies likes rumen epithelium damage and ruminitis, metabolic acidosis, respiratory acidosis, liver abscesses, ketosis, laminitis, increased bloat, and abomasal dysplasia among others. The embodiments disclosed include systems, devices, and methods to focus enquiry on the microbial biomass increase and increase in carbon dioxide at the first state of acidosis, and on the increased fermentation solvents on the second state of acidosis.

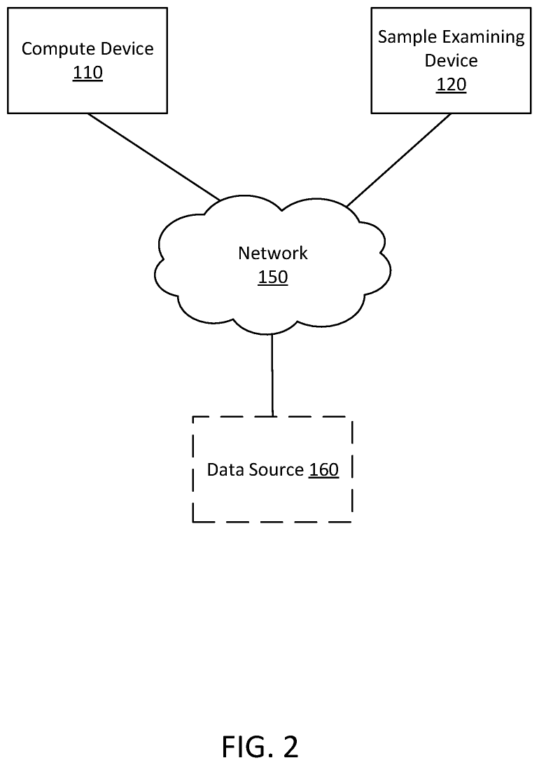

FIG. 2 is a high-level block diagram that illustrates an acidosis detection system (also referred to herein as ADS or “the system”) 100 for processing samples of bodily fluid or gas from an oral or nasal cavity of a ruminant, according to some embodiments. The system can be configured to receive the sample, process the sample for the presence of one or more identified solvents or components, and to provide an output indicating the presence and/or a measured amount of the identified solvent or component. The system 100 can include a compute device 110, a sample examining device 120, and optionally a data source 160. In some embodiments, the compute device 110 can communicate with the sampling device 120 and optionally with the data source 160, to perform sample processing and sample analysis, acidosis detection, and optionally any suitable health prediction or feed planning to provide computer generated guidance to an animal health specialist or livestock manager during maintenance and treatment of livestock like ruminants (e.g., dairy cows for yielding milk).

In some embodiments, the sample examining device 120 (“SED” or “SE device”) may be configured to receive and/or extract a sample of bodily fluid or gas from an animal, process the sample and perform acidosis detection. The device can be configured to indicate a presence of an identified solvent. In some embodiments, the device can be configured to measure a quantity of the identified solvent present in the sample. The SE device 120 can be configured indicate the presence and/or the measured quantity of the identified solvent or component. In some embodiments, the SE device 120 can be configured to transmit an output, for example, to a compute device 110.

In some embodiments, the compute device 110 may be configured to perform acidosis detection to detect and identify one or more solvents or components in a bodily fluid from an animal. The compute device 110 can be configured to process the solvent to identify characteristics associated with the solvent indicating a state of acidosis. For example, the compute device 110 can identify a concentration of the identified solvent in the sample analyzed, a type of solvent detected in the sample analyzed, and/or the like. In some embodiments, the compute device 110 can be configured to perform a health prediction based on the detection of solvent and/or a state of acidosis. For example, the compute device 110 can be configured to generate output indicating an expected progression of the state of acidosis if left unchecked, a prediction of interventions, medical and/or feed supplements, and/or treatments that can be used to ameliorate or treat the animal to a state of recovery from the state of acidosis, and/or the like. In some embodiments, the compute device 110 can be configured to generate a feed plan or a feed management plan to help set the animal on a path to recovery from the state of acidosis.

The compute device 110 may be implemented as a single compute device, or be implemented across multiple compute devices that are connected to each other and/or the network 150. For example, the compute device 110 may include one or more compute devices such as mobile phones, servers, desktop computers, laptop computers, portable devices, databases, etc. Different compute device may include component(s) that are remotely situated from other compute devices, located on premises near other compute devices, and/or integrated together with other compute devices.

In some embodiments, the compute device 110 can be located on a server that is remotely situated from one or more imaging device(s) 160 and/or image data storage 180. In some embodiments, the compute device 110 can be integrated into one or both of the sample examining device 120 and/or the data source 160. In some embodiments, the compute device 110 can be integrated into a sample examining device 120 (for example, a sample examining device that is configured to be a solvent analyzer integrated with the compute device 110). In some embodiments, system 100 includes a single device that includes the functionality of the compute device 110, the sample examining device 120, and the data source 160, as further described herein.

In some embodiments, the compute device 110 can be located within an animal health facility, a farm or other livestock management facility, or an animal husbandry facility. The compute device 110 can be operatively coupled to one or more data sources 160 associated with the animal health facility, e.g., a veterinary hospital database or a livestock management database for storing animal health information, animal feed information, etc. In some embodiments, the compute device 110 can be available to farmers, farm hands (e.g., feeding personnel, animal health monitoring personnel), veterinary physicians, and livestock management personnel for performing evaluation of animals and/or for monitoring animals for a health status (for example, pathophysiologies like acidosis, ruminitis, etc. as described herein), visualization of animal health data, diagnoses, and/or planning of livestock management, yield expectation, and/or the like. In some embodiments, the compute device 110 can be operatively coupled to one or more other compute devices within a hospital (e.g., a veterinarian workstation), and can send outputs and/or other data to such compute devices (e.g., via network 150) for visualization of animal health data, diagnoses, performing evaluation of animal health over time, recommend feed supplements and/or medical supplements, and/or planning of yield harvesting and ongoing livestock maintenance.

Network 150 may be any type of network (e.g., a local area network (LAN), a wide area network (WAN), a virtual network, a telecommunications network) implemented as a wired network and/or wireless network and used to operatively couple compute devices, including system 100. As shown in FIG. 1, a connection may be defined between compute device 110, the sample examining device 120, optional data source 160, and/or other compute devices (e.g., databases, servers, etc. that are shown in FIG. 1). In some embodiments, the compute device 110 may communicate with SE device 120 and/or data source 160 (e.g., send data to and/or receive data from such devices) and with the network 150 via intermediate networks and/or alternate networks (not shown in FIG. 1). Such intermediate networks and/or alternate networks may be of a same type and/or a different type of network as network 150. Each of the compute device 110, SE device 120, and data source 160 may be any type of device configured to send data over the network 150 to send and/or receive data from one or more of the other devices.

In some embodiments, a data source 160 may refer to any device configured to obtain and/or store data associated with an identified animal including for example animal health (e.g., a history of health status in an animal in a managed cohort, a history of intake, biomass extracted from the animal, results from any medical, physical, and/or other examinations of the animal), a history of feed supplements, medical supplements, and/or medical interventions provided to an identified animal in a cohort), a data associated with a yield or production of a bioproduct (e.g., milk, meat, etc.) associated with an identified animal, properties of a bioproduct obtained from an identified animal (e.g., a fat content associated with milk obtained from an animal, a protein content associated with milk obtained from an animal, amount of milk fat content in the milk, amount of milk protein content in the milk, microbial abundance in an animal sample, etc.). A data source 160 be configured to obtain and/or store data associated with examination of a sample from an identified animal including for example animal health status of that animal at various points of examination, properties of solvents or components associated with the animal health status, data associated with synthetic microbial ensembles including one or more identified microorganism strains that may be used to address diagnoses in an animal, one or more identified carriers that can be configured to absorb one or more solvents associated with a health status of an animal, one or more microbial growth inhibitors configured to inhibit grown of identified microbes and/or inhibit solventogenesis in an animal, and/or the like.

In some embodiments, the data source 160 may associated with a laboratory or analysis provider that is used to perform biochemical analysis, imaging analysis, surgical analysis, tubing studies, and/or the like of samples from identified animals.

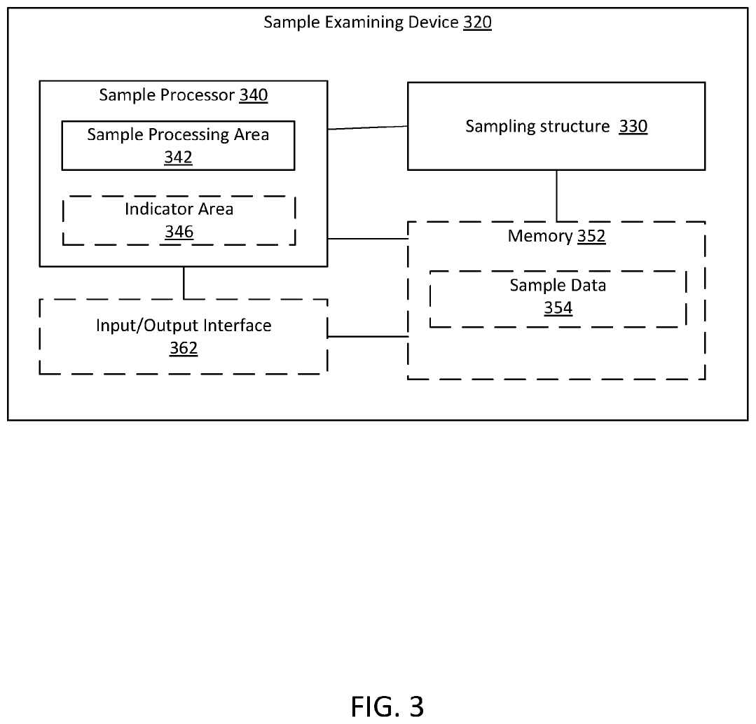

FIG. 3 schematically illustrates an SE device 320 that can be a part off an acidosis detection system like the system 100 shown in FIG. 2. The SE device 320 can be substantially similar in structure and/or function to the SE device 120 described with reference to the system 100 in FIG. 2. The SE device 320 includes a sampling structure 330, a sample processor 340, optionally a memory 352 and optionally an input/output interface 362. The sample examining device 320 is configured to obtain a sample of a bodily fluid from a nasal cavity or an oral cavity of an animal and process the sample. In some embodiments, the SE device 320 can be configured to capture a bodily sample are to extract a bodily sample from an animal from its oral or nasal cavity. In some embodiments, the SE device 320 can be configured to process the sample and to indicate a result associated with the sample. For example, the SE device 320 can be configured to process the obtained sample of bodily fluid to detect a presence of a solvent associated with a pathophysiology for example, acidosis. In some embodiments the SE device 320 can be configured to indicate the presence or absence off an identified solvent, for example ethanol, or an identified gas, for example carbon dioxide, in the obtained sample via an indicator area. In some embodiments, the SE device 320 can be configured to process the sample to measure a property associated with an identified solvent, for example, an amount, a quantity, a concentration, and/or the like.

In some embodiments the SE device 320 can be an apparatus like a test strip configured to capture a sample in a sampling structure and process the sample in a sample processing are through a sample testing process to provide an indication off a presence/absence and/or a property associated with an identified solvent. The apparatus or test strip can be configured to test for a target analyte which can be an identified solvent associated with a pathophysiology like acidosis.

The sampling structure 330 can be any suitable structure configured to receive a sample of bodily fluid or gas to be analyzed for the presence of, absence of, and/or one or more properties associated with an identified solvent (e.g., ethanol). The bodily fluid can be any suitable fluid obtained from a nasal cavity or an oral cavity of a ruminant. For example, the bodily fluid can be saliva, nasal secretions, buccal or oral secretions, and/or the like. The gas can be exhaled air from a respiratory system of an animal obtained from a nasal cavity of the animal.

The sampling structure 330 can be configured to receive any suitable amount of bodily fluid or gas. (e.g., 1 μl, 2 μl, 5 μl, 10 μl, 15 μl, 20 μl, 25 μl, 50 μl, 100 μl, 250 μl, 500 μl, 1 ml, 5 ml, 10 ml, 25 ml, 50 ml, 100 ml, 500 ml, and/or any intermediate amount therein). In some embodiments, the sampling structure 330 can be specially adapted to capture the sample (e.g., a bodily air) without allowing it to dissipate. In some embodiments, the sampling structure 330 can be a structure, material, chamber, cavity, receptacle, or any other suitable component adapted to receive a bodily sample of a defined volume or quantity. The sampling structure 330 can be configured to convey the sample or a portion of the sample to the sample processor 340. For example, the sampling structure can include a region or material or structure adapted to receive a sample of bodily fluid and to convey the sample or a portion of the sample to a sample processing area in a sample processor 340.

The sample processor 340 includes a sample processing area 342 configured to process the sample or portion of sample conveyed from the sampling structure via any suitable sample testing process to detect the presences of an identified solvent and/or to measure a property associated with the identified solvent. In some embodiments, the sample processing area 342 can include one or more adaptations (e.g., materials, structures, etc.) configured to conduct a sample testing process to probe the sample or portion of sample conveyed from the sampling structure for the presence, absence, and/or one or more properties of an identified solvent.

The identified solvent can be any suitable solvent associated with a state of acidosis in a ruminant. For example, the solvent can include an alcohol including ethanol, propanol, isopropanol, butanol, acetone, acetaldehyde, succinate, and/or the like. While the present disclosure describes solvents associated with acidosis, it can be appreciated that the disclosed systems, devices, and methods can be used to detect and/or measure properties associated with any suitable solvent associated with any suitable pathophysiology associated with a rumen of a ruminant, including pathophysiology likes rumen epithelium damage and ruminitis, metabolic acidosis, respiratory acidosis, liver abscesses, ketosis, laminitis, increased bloat, and abomasal dysplasia among others. In some embodiments, the sample processing area 342 can be adapted to conduct a suitable sample testing process to detect a presence of an identified solvent and/or to determine a property associated with an identified solvent (e.g., a quantity, a volume, a concentration, and/or the like).

The sample testing process can be any suitable testing process including an enzymatic assay, a chromatographic test, an immunoassay, a colorimetric assay, and/or the like. The sample processing area 342 can be specially adapted to conduct the sample testing process. For example, the sample processing are 342 can includes one or more test reagents used for conducting the sample testing process. The test reagent can be localized in the sample processing are 342 such that when the sample or portion of sample to be tested is conveyed to the sample processing area the test reagent reacts with the portion of the sample. The test reagent can be configured to undergo a change or a transformation if it comes in contact with the identified solvent for a predetermined period of reaction time, such that the change or transformation from a first state to a second state can be detected to infer a presence. In some embodiments, the test reagent can be configured to undergo one or more transformations the resulting state indicating not only a presence of a solvent but also a property associated with the solvent (e.g., a concentration).

In some embodiments, optionally the sample processor 340 can be configured to define an indicator area 346 designated to provide a region where the change or transformation of the reagent(s) can be easily represented for a user to read a result of processing the sample. The change or transformation can be exhibited or can be represented in any suitable form. For example, the change or transformation can be represented in a change in color that is visibly detectable, or detectable under light of a suitable wavelength, etc. In some embodiments, the sample processing area 342 and the indicator are 346 can be configured to conduct a testing process and to provide results from the testing in the indicator area 346 for easy access to the results of sample processing.

In some embodiments, the sample processing area 342 and the indicator area 346 can be configured to include a reagent that is configured to undergo a first change when a certain first threshold criterion is met and a certain second change when a certain second threshold criterion is met. For example, the reagent can undergo a first change or transformation (e.g., emitting or causing an emission of a first color) when a first criterion of a mere presence of a solvent is met. The reagent can undergo a second change or transformation (e.g., emitting or causing an emission of a second color different than the first color) when a second criterion of a presence of a first threshold concentration of the solvent is met. The reagent can undergo a third change or transformation (e.g., emitting or causing an emission of a third color different than the first and second colors) when a third criterion of a presence of a second threshold concentration of the solvent is met, and so on.

In some embodiments, the sample processing area and the indicator are 346 can be configured to include multiple reagents each reagent configured to undergo a change when a certain threshold criterion is met, for example, a first reagent having a first criterion of a mere presence of a solvent, a second reagent having a second criterion of a presence of a first threshold concentration of the solvent, a third reagent having a third criterion of a presence of a second threshold concentration of the solvent that is greater than the first threshold concentration, and so on.

The testing process can be suitably selected and the sample processing area 342 and the indicator area 346 of the sample processor 340 can be suitably configured such that a wide range of properties of solvents can be detected and/or indicated. For example, in some embodiments, the sample processor 340 can be configured to detect any concentration between 0 and 50 mM of an identified solvent, for example ethanol. In some embodiments, the sample processor 340 can be configured to measure and indicate a presence of a concentration of the solvent within a predetermined window e.g., between 0 and 1 mM, between 1 mM and 5 mM (e.g., daily average measurements can range between 1 mM and 5 mM), 1 mM-10 mM, 10-20mM, 20-30 mM, 30-40 mM, 40-50 mM, and so on. In some instances, a concentration beyond 10 mM can be indicative of a symptom or an extreme symptom associated with a health status of the animal. In some embodiments, the sample processor 340 can be configured to indicate a lower threshold of concentration of solvent detected and measure. For example, indicate if the concentration of solvent detected is greater than 1 mM, 5 mM, 10 mM, 25 mM, 35 mM, 50 mM, and so on (including all intermediary values in between the example illustrated here). While described herein as adapting a testing process to detect presence or a concentration of an identified solvent (e.g., ethanol), it can be appreciated that embodiments of the sample examining device 320 can include a sample processor 340 suitably adapted (e.g., a sample processing area and/or an indicator area suitably adapted) to detect and/or indicate a presence of an identified solvent and measure the presence using any suitable property like quantity, mass, volume, pH associated with the solvent.

In some embodiments, as it may be appreciated, the sample processing area 342 can have any suitable adaptation to test the portion of sample for any property including biochemical, electrochemical, metabolic, electrolytic, conductive, thermal, enzymatic, an/or immunological property that may be used to detect a presence of an identified solvent or gas and/or measure a property associated with the identified solvent or gas.

In some embodiments, the sample processes or 340 in a sample examining device 320 can include one or more processors (not shown in FIG. 3) configured to coordinate a sampling function of the sampling structure 330, and/or a sample processing function, a conducting the testing process function, and/or result indicating function of the sample processor 340. For example, a processor can be configured to aid, initiate, or cause a sample or a portion of sample to be conveyed from the sampling structure 330 to the sample processor, cause the testing process and the indication of result by the sample processor. The processor may be any suitable processing device configured to run and/or execute any of the functions described herein. In some embodiments, the processor may be a general purpose processor, a Field Programmable Gate Array (FPGA), an Application Specific Integrated Circuit (ASIC), a Digital Signal Processor (DSP), a Dedicated Graphics Processing Unit (GPU), and/or the like. In some embodiments, the processor can be configured to implement one or more of the functions described herein as one or more programs and/or applications that are tied to hardware components (e.g., processor, memory 352, input/output interface(s) 362). In some embodiments, a system bus (not shown) may be configured to enable the sampling structure 330, sample processor 340, memory 352, input/output interface(s) 362, and/or other components of the SE device 320 to communicate with each other.

In some embodiments, the sample examining device 320 optionally includes an input/output interface 362, configured to receive transmit an output associated with the sample testing process described herein. For example, in some embodiments, the sample examining device 320 can be configured such that a result associated with the sample testing process conducted in the sample processor 340 can be electronically transmitted to an external device such as a compute device (e.g., the compute device 110 of system 100) or a data source (e.g., data source 160 of system 100).

The input/output interface(s) 362 may include one or more components that are configured to receive inputs and send outputs to other devices (e.g., compute device 110, data source 160 of system 100, etc.). In some embodiments, the input/output interface(s) 362 can include a user interface, which can include one or more components that are configured to receive input and/or present output to a user. For example, input/output interface 362 may include a display device (e.g., a display, a touch screen, etc.), an audio device (e.g., a microphone, a speaker), a keypad, and/or other interfaces for receiving information from and/or presenting information to users. In some embodiments, the input/output interface 362 can include a communications interface for communicating with other devices, and can include conventional electronics for data communication using a standard communication protocol, e.g., Wi-Fi, Bluetooth®, etc.

In some embodiments, the SE device 320 optionally includes a memory 352 operatively coupled to the sampling structure 330, the sample processor 340, and the optional input/output interface 3662. In some embodiments, the memory 352 can be configured to store data and/or instructions used to conduct the sample acquisition by the sampling structure 330, the sample testing and result indication by the sample processor 340, and/or the input acquisition and/or output transmission by the input/output interface 362. The memory 352 can be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, memory 352 stores instructions that causes a processor associated with the SE device 320 to execute modules, processes, and/or functions associated with sample acquisition, sample processing, result indication and/or the like. Memory 352 can store sample data 354 which can include details or information associated with a sample tested (e.g., a source bodily fluid from which the sample was obtained, identity of an animal from which the sample was obtained, data associated with the source (e.g., a health status of the animal at the time of testing, a diet associated with the animal at time of testing, a phase of livestock management that the animal was part of at time of testing such as transition phase of dairy cows, calving phase of dairy cows, a post-calving, negative energy balance phase for dairy cows, post-calving acclimatization phase with an identified milk yield, etc.), properties of the sample (e.g., date of collection, method of collection, amount used for testing, test used, the identified solvent that the sample is being tested for, data associated with a pathology suspected or indicated, results from the testing, and/or the like.

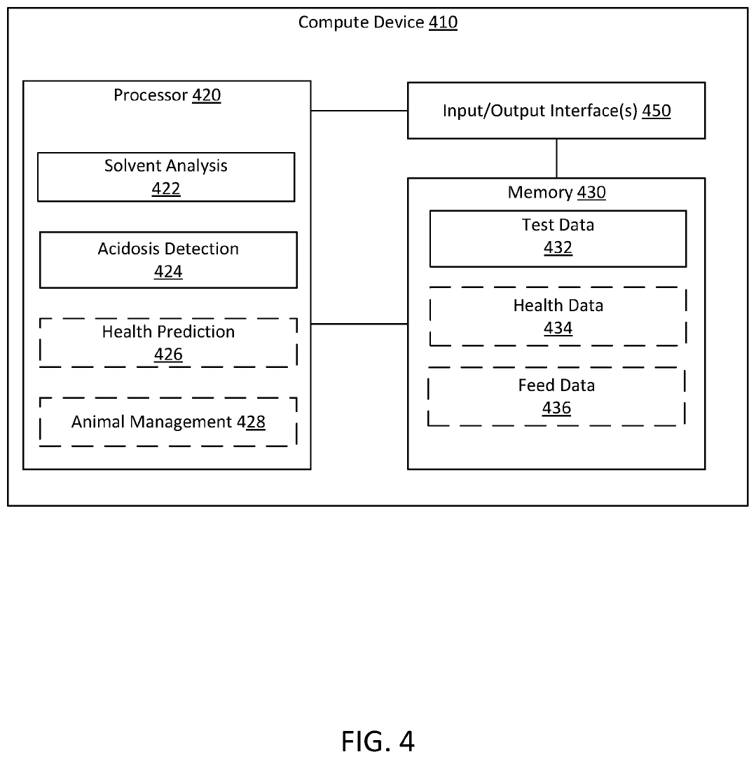

FIG. 4 schematically illustrates an example compute device 410 for acidosis detection, according to some embodiments. Compute device 410 can be structurally and/or functionally similar to compute device 110. While a single compute device 410 is schematically depicted, it can be appreciated that the compute device 410 can be implemented as one or more compute devices. In some embodiments, compute device 410 may be configured to analyze a presence or a measured property of a solvent in a sample of bodily fluid or gas from an animal, detect a state of acidosis, predict a health status of an animal at a time of testing and following a time of testing depending on other parameters like health, medical intervention and/or supplementation, monitoring, and/or feeding, and/or generate a plan or recommendation for animal management including supplementation, feed alteration, and/or medical intervention. Compute device 410 includes a processor 420, a memory 430, and one or more input/output interface(s) 450.

Memory 430 may be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, memory 430 stores instructions that cause processor 420 to execute modules, processes, and/or functions associated with solvent analysis 422, acidosis detection 424, and optionally health prediction 426, and/or animal management 428. Memory 430 can store one or more test data 432, and optionally health data 434, and/or feed data 436. Test data 434 can include information associated with samples processed by a sample examining device (e.g., SE device 120, 320) to perform the solvent analysis 422 and acidosis detection 424, and optionally the health prediction 426, and/or animal management 428. Health data 434 and feed data 436 can be used to perform health prediction 426 and/or animal management 428.

In some implementations, health data 434 and/or feed data 436 can include data obtained from a data source e.g., data source 260 of system 100 described with reference to FIG. 2 including datasets obtained from farms, research facilities, testing laboratories, veterinary hospitals, feed or medical supplement suppliers or providers, regulatory agencies overseeing animal feed and health management (e.g., The Association of American Feed Control Officials (AAFCO), and/or the like.

In some embodiments, health data 434 can include one or more physical health parameters of an animal in a cohort (e.g., age, temperature, respiratory state, blood, or serum analysis, etc.) a diet of the animal, a feed uptake by the animal, living space, condition of its pen or holding chamber, rationing of feed, feed mixing, mechanism or means of feed delivery, water availability, drinking behavior, feed intake, intake schedule, frequency, etc. A health status can include a physical state of an animal in the context of animal or livestock management. For example, a phase of livestock management associated with a dairy cow such as a pregnancy phase, transition phase, calving phase, a post-calving, negative energy balance phase, post-calving acclimatization phase with an identified milk yield, and/or the like). In some implementations, health data can include data related to a quantity/quality of bioproduct, for example, milk, produced by an animal. For example, health data 436 can include an indication of milk yield, an amount of fat in milk, an amount of protein in milk, a quantity milk fat and/or milk protein, a number of days producing milk, an average rate of milk production, an amount of dry extract in milk, a number of liters of milk per day, an amount of urea in milk, a bacterial biomass associated with a bodily fluid tested from the cow, bacterial count in milk, inhibitors in milk, an amount of casein in milk, and/or the like. In some embodiments, an indication of health status can include an indication of a pathology onset or a risk of imminent pathology onset, or an on going pathology, medical interventions and/or feed supplements provided, schedule of medical interventions and/or feed supplements, and/or the like.

Feed data 436 can include a range of diets used or potentially could be used for an animal in a given cohort or in a given health status. Feed data 436 can include data associated with feed composition, feeding schedule, feeding mechanism, watering schedule, history of feeding, feed supplements available, history of feed supplements used and the resulting changes in health of an animal, and/or the like. In some embodiments, the feed data 436 can include information associated with potential carriers that can be used as a feed supplement including Zeolite, charcoal, activated granulated charcoal, bone ash, bone charcoal, diatomaceous earth, kaolin, bentonite, and clay. In some embodiments, charcoals associated with various properties can be suitably used as potential carriers. Some properties of interest can include porosity, surface area (e.g., component having more surface area can be used to absorb more solvent) and ash content (e.g., a component having less ash content can be selected to have better absorptive ability). In some implementations, powdered charcoal and/or granulated charcoal can be selected to use as carriers. IN some implementations, granulated activated charcoal can show improved performance than for example, powdered charcoal (e.g., sometimes because the ash content of powdered charcoal can be greater than granulated charcoal). In some embodiments, tests can be performed to compare the performance or efficacy of two or more materials and the results can be used to suitably select one or more carriers to use. In some embodiments, the feed data 436 can include information associated with potential microbial growth inhibitors that can be included with feed to provide an animal to induce a change in a health status. As an example, potential microbial growth inhibitors can include ascorbic acid, benzoic acid, propionic acid, sodium sulfite, sulfur dioxide, sodium nitrite, and fumaric acid.

The processor 420 may be any suitable processing device configured to run and/or execute any of the functions described herein. In some embodiments, processor 420 may be a general purpose processor, a Field Programmable Gate Array (FPGA), an Application Specific Integrated Circuit (ASIC), a Digital Signal Processor (DSP), a Dedicated Graphics Processing Unit (GPU), and/or the like. In some embodiments, the processor 420 can be configured to perform one or more of solvent analysis 422, acidosis detection 424, optionally health prediction 426 and animal management 428. The processes of solvent analysis 422, acidosis detection 424, optionally health prediction 426 and animal management 428 can be implemented as one or more programs and/or applications that are tied to hardware components (e.g., processor 420, memory 430, input/output interface(s) 450). In some embodiments, a system bus (not shown) may be configured to enable processor 420, memory 430, input/output interface(s) 450, and/or other components of the compute device 410 to communicate with each other.

While a single processor 420 that is located on a single compute device 410 is depicted in FIG. 4, it can be appreciated that processor 420 can be one or more processors that are located on the same compute device and/or different compute devices. In some embodiments, systems, devices, and methods described herein, including, for example, solvent analysis 422, acidosis detection 424, optionally health prediction 426 and animal management 428 can be implemented in a cloud platform (e.g., using one or more remote compute devices). The cloud platform can be connected to one or more databases, such as, for example, farm and/or veterinary hospital databases, via a network (e.g., network 150). As such, systems, devices, and methods described herein can receive information from those databases (e.g., animal health information, acidosis detection test data, feed data, etc.) and/or send information to those databases (e.g., acidosis detection results, amount or concentration of solvent measured in a sample of biological fluid, health status of animal, yield, or properties of bioproduct yielded by an animal, etc.)

The input/output interface(s) 450 may include one or more components that are configured to receive inputs and send outputs to other devices (e.g., data source(s) 160, Se device(s) 120, 320, etc.). In some embodiments, the input/output interface(s) 450 can include a user interface, which can include one or more components that are configured to receive input and/or present output to a user. For example, input/output interface 450 may include a display device (e.g., a display, a touch screen, etc.), an audio device (e.g., a microphone, a speaker), a keypad, and/or other interfaces for receiving information from and/or presenting information to users. In some embodiments, the input/output interface 450 can include a communications interface for communicating with other devices, and can include conventional electronics for data communication using a standard communication protocol, e.g., Wi-Fi, Bluetooth®, etc.

### 2. Methods

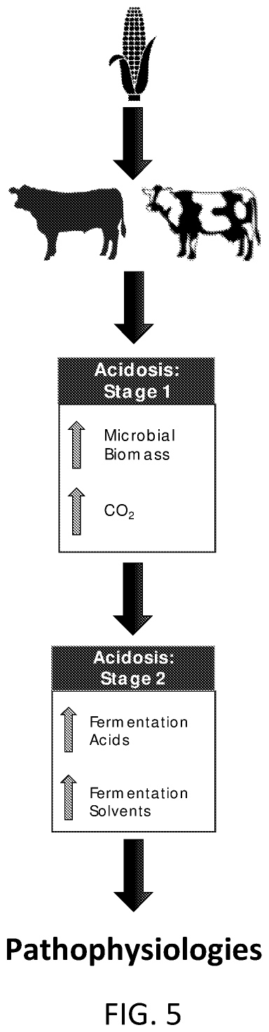

Systems, devices, and methods described herein can perform detection of an identified solvent or gas in a sample of bodily fluid obtained from a nasal or oral cavity of an animal, measurement of a property associated with the detected solvent or gas, and based optionally based on the detection of solvent and/or measurement of the property associated with the solvent or gas determine a health status, such as acidosis, of the animal from which the sample was obtained. In some embodiments, systems and devices and methods described herein can perform such solvent measurement/analysis, acidosis detection, in combination with health prediction and animal management, as described herein. FIG. 5 illustrates a progression of acidosis in ruminants including an acidosis stage 1 that is indicated by an increase in microbial biomass and increase in carbon dioxide, followed by an acidosis stage 2 indicated by an increase in fermentation acids and increase in fermentation solvents. Acidosis stage 2 is followed by pathophysiologies described herein In some embodiments, the methods and systems described herein can be used to test samples of bodily fluids to detect a health status that can include a state of healthfulness, a state of risk of onset of acidosis, a state of onset of acidosis stage 1, a state of acidosis stage 2, and/or a state of pathophysiology past the acidosis stage 2.

As described above, a compute device (e.g., compute devices 110, 410) for performing solvent measurement/analysis, acidosis detection, health prediction, and/or animal management can implement one or more algorithms, routines, analyses, calculations, and/or models. In some embodiments, the algorithms or models can include statistical, mathematical, and/or machine learning models or tools, which can be trained using labeled training datasets and/or validated using designated validation datasets.

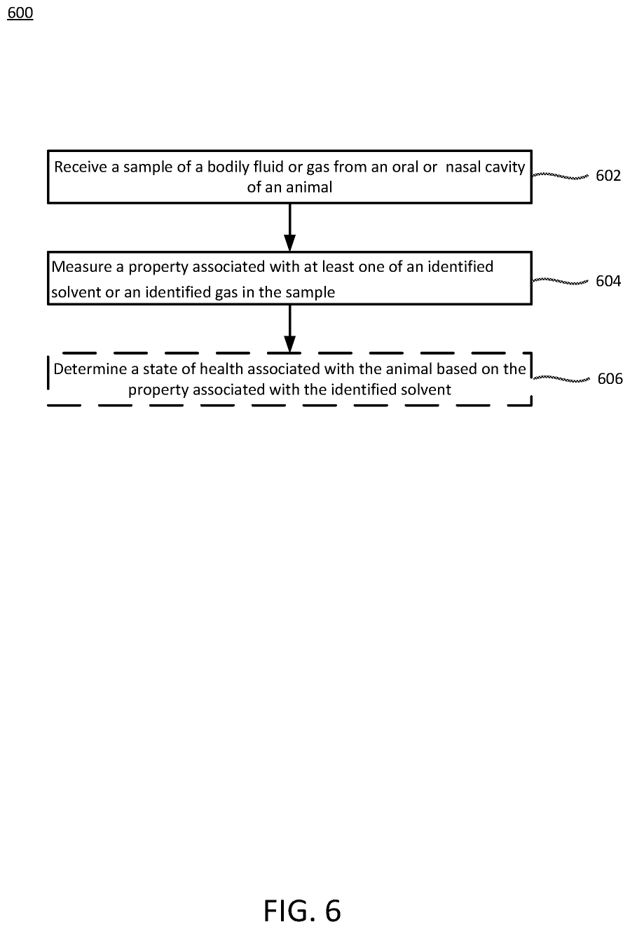

In some embodiments, an ADS (e.g., system 100) can perform solvent detection and/or measurement of a property associated with a solvent from a sample of bodily fluid using a sample examining device. FIG. 6 is a flow chart illustrating an example method 600 of determining a state of health of an animal using an ADS, according to an implementation. The method 600 can be performed by any ADS described herein, e.g., ADS 100 of FIG. 2, and/or any of the sample examining devices and/or compute devices thereof. For example, the method 600 can be implemented using an SE device such as the SE device 320 of FIG. 3. In some implementations, the method 600 can be implemented by a combination of an SE device (e.g., SE device 320) and a compute device (e.g., compute device 110 of FIG. 1 and/or compute device 410 of FIG. 4.

As shown in FIG. 6, the method 600 includes receiving a sample pf a bodily fluid or gas from an oral or nasal cavity of an animal at 602. As described herein, the bodily fluid can be any suitable bodily fluid including saliva, nasal secretions, buccal secretions, and/or any suitable bodily fluid of oronasopharyngeal origin. The sample can be received in a sample examining device (e.g., SE device 320), for example in a sampling structure. In some example embodiments, the SE device can be configured to receive a sample in a sampling structure adapted to receive the sample, contain the sample, and convey the sample to a sample processor. In some example embodiments, the SE device can be a test kit or a test strip of a material (e.g., absorbent, or sieving material) including a sampling structure configured to receive a sample to be tested and convey the sample to a sample processing area.

The method 600, at 604, includes measuring a property associated with at least one of an identified solvent or an identified gas in the sample. In some embodiments, the SE device can be configured such that a sample processor includes a test reagent configured to react with a sample containing the identified solvent or gas. The sample processor can be configured to receive the sample or a portion of the sample, from the sampling structure, and conduct or cause the conduction of a testing process, i.e., initiate or cause a reaction of the test reagent with the identified solvent or gas, if present in the portion of sample. In some embodiments, the SE device can be a test kit or test strip that includes a defined sample processing area that includes a reagent configured to react with the identified solvent, for example, ethanol (or gas, e.g., carbon dioxide). defined a sample processor configured to conduct or cause the conduction of a testing process when the sample is conveyed to the sample processor, and an indicator area configured to provide or indicate a result of the testing process, i.e., indicate a presence/absence of an identified solvent or gas and/or a measurement of a property associated with the identified solvent or gas. For example, the solvent can be ethanol and the sample processor can be configured to measure a concentration of ethanol detected in the sample. In some embodiments, the sample processor can be configured to cause a change or transformation in a regent that causes an indication of the measure concentration. The testing process can be any suitable testing process (e.g., an enzymatic assay, a chromatographic test, an immunoassay, a colorimetric assay, a peroxidase based assay an/or the like). The change or transformation can be indicated in any suitable forma. For example, through an appearance or disappearance of one or more visible markings (e.g., in an indicator area), a color change, a fluorescent emission, and/or the like. In some embodiments the sample processor can be configured to measure a concentration of ethanol as low as 0 or 0.001 mM and to measurements of around 1.5 mM-10 mM (e.g., daily measurements can fluctuate between 2 mM-5 mM) to up to 30 mM, 40 mM, and/or 50 mM.

In some embodiments, the testing process can be such that the reagent is configured to undergo a first change when a certain first threshold criterion is met and/or a certain second change when a certain second threshold criterion is met. For example, the reagent can undergo a first change or transformation (e.g., emitting or causing an emission of a first color) when a first criterion of a mere presence of a solvent is met but when the concentration of the solvent is below a first threshold concentration (i.e., presence is mere trace amounts or is comparable to a baseline concentration) greater than the first threshold. The reagent can undergo a second change or transformation (e.g., emitting or causing an emission of a second color different than the first color) when a second criterion of a presence of a first threshold concentration of the solvent is met. The reagent can undergo a third change or transformation (e.g., emitting or causing an emission of a third color different than the first and second colors) when a third criterion of a presence of a second threshold concentration of the solvent is met, and so on. In some embodiments, the reagent can be configured to transform incrementally with increase in concentration. In some embodiments, the reagent can be configured to under the change or transformation corresponding the concentration of the solvent regardless of any lower threshold criteria.

In some embodiments, the sample processing area and the indicator are can be configured to include multiple reagents each reagent configured to undergo a change when a certain threshold criterion is met, for example, a first reagent having a first criterion of a mere presence of a solvent, a second reagent having a second criterion of a presence of a first threshold concentration of the solvent, a third reagent having a third criterion of a presence of a second threshold concentration of the solvent that is greater than the first threshold concentration, and so on.

As described herein, the testing process can be suitably selected and the sample examining device can be suitably configured such that a wide range of properties of solvents can be detected and/or indicated. For example, in some embodiments, the solvent can be ethanol and any concentration between 0 and 50 mM of ethanol can be detected, measured and/or indicated. In some embodiments, the method 604 can include detecting and measuring a presence of a concentration of ethanol within a predetermined window e.g., between 0 and 1, 1 and 1.5 Mm, 1 and 2 mM, 1 and 5 mM, 2-5 mM, 5-8 mM, 8-10 mM, 1-10 mM, 1-20 mM, 10-20 mM, 20-30 mM, 30-40 mM, 40-50 mM, and so on. In some embodiments, the method 600 at 604 can include indicating a lower threshold of concentration of ethanol detected and measured. For example, the method can include indicating if the concentration of ethanol detected is greater than 1 mM, 5 mM, 10 mM, 25 mM, 35 mM, 50 mM, and so on (including all intermediary values in between the example illustrated here). While described to measure a concentration of ethanol, for illustration purposes, in some implementations, the method 600 at 604 can be suitably used to measure any suitable property like quantity, mass, volume, pH associated with any suitable solvent including ethanol, propanol, isopropanol, butanol, acetone, acetaldehyde, succinate, and/or the like.

The method 600 at 606 includes determining a state of health associated with the animal based on the property associated with the identified solvent. In some implementations, the ADS can utilize a compute device (e.g., compute device 110, 410 to determine a state of health associated with an animal from an indication of a property associated with an identified solvent (e.g., concentration of ethanol) in a bodily fluid like saliva obtained from the animal.

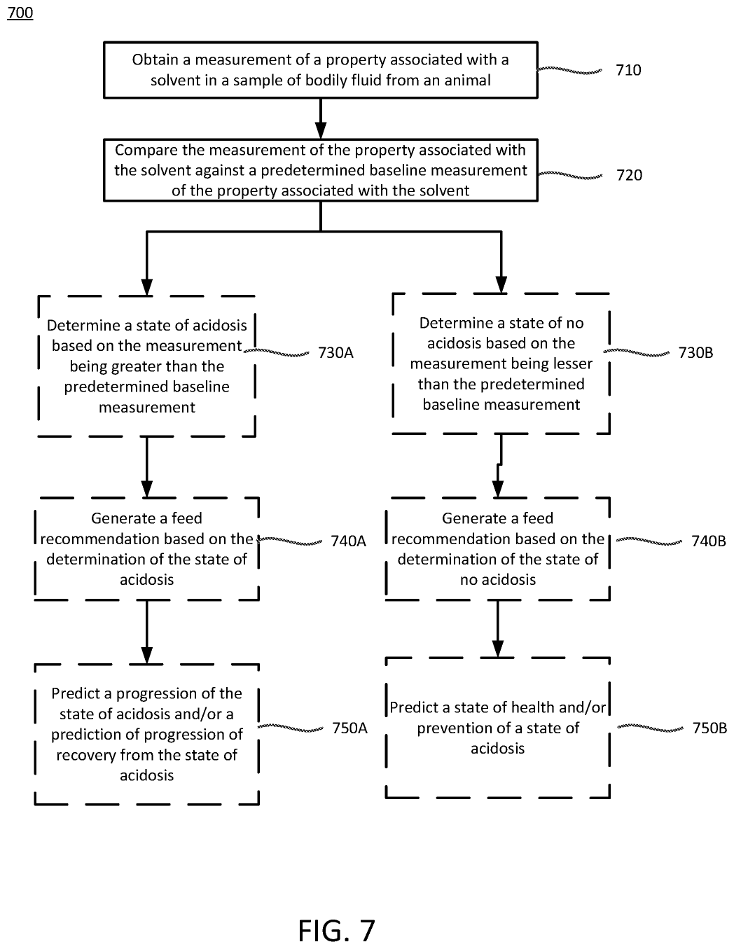

FIG. 7 is a flow chart illustrating an example method 700 of determining a state of health of an animal based on information associated with a solvent in a sample of bodily fluid from the animal, using an ADS, according to an implementation. The method 700 can be performed by any ADS described herein, e.g., ADS 100 of FIG. 2, and/or any of the compute devices thereof (e.g., compute device 110, 410).

As shown in FIG. 7, the method 1000 includes, at 710, obtaining a measurement of a property associated with a solvent in a sample of bodily fluid from an animal. For example, obtaining a concentration of a solvent ethanol in a sample of saliva from a dairy cow. At 720, the method includes comparing the measurement of the property associated with the solvent against a predetermined baseline measurement of the property associated with the solvent. As an example, the measured concentration of ethanol can be a test measurement. The ADS can obtain a predetermined baseline measurement of ethanol that is expected in a saliva of a dairy cow that is known to be healthy (without any known pathophysiology). The ADS can be used to compare the test measurement against the predetermined baseline measurement of ethanol in the saliva of a dairy cow. In some implementations, the method can include comparison against more than one predetermined measurements as described herein. For example, in some implementations, the method can include comparing a test measurement against a predetermined lower threshold or a baseline measurement or a negative control measurement, as well as an upper threshold or a positive control measurement, or a measurement of concentration of ethanol from a saliva of a dairy cow known to have a state of acidosis (e.g., acidosis stage 1, acidosis stage 2, pathophysiology, etc.).

The comparison at 720 can result in one of two outcomes. Either the test measurement can indicate a statistically higher concentration of ethanol compared to the predetermined baseline measurement, or the test measurement can be statistically comparable concentration of ethanol compared to the predetermined baseline measurement.

At 730A, the method 700 includes determining a state of acidosis based on the measurement being greater than the predetermined baseline measurement. In some implementations, the method can include more than one predetermined baseline measurements to determine more than one state of acidosis. For example, the method at 730A can include determining a first state of acidosis (e.g., an acidosis stage 1) based on a test measurement of concentration of ethanol being greater than a first predetermined baseline measurement but the test measurement being less than a second predetermined positive control measurement associated with a stage further progressed than acidosis stage 1. The method at 730A can include determining a second state of acidosis (e.g., an acidosis stage 2) based on a test measurement of concentration of ethanol being greater than a first predetermined baseline measurement and also greater that the second predetermined positive control measurement associated with a stage further progressed than acidosis stage 1, for example, acidosis stage 2, and so on.

Following 730A, the method includes, at 740A, generating a feed recommendation based on the determination of the state of acidosis at 730A. And at 750A the method includes predicting a progression of the state of acidosis and/or a prediction of progression of recovery from the state of acidosis. In some implementations, an ADS can be used to generate a health prediction for example an expected progression of the acidosis. In some implementation, the method can include recommending a feed supplement or a change in diet to counter the effects of acidosis based on the determination at 730A. For example, in some implementations, an ADS can be used (implementing a health prediction and/or an animal management process described herein) to recommend addition of (i) a synthetic microbial ensemble including one or more identified microorganism strains (e.g., one or more microbes described in sections below with reference to Tables 1, 2, 3, 4, 5, and/or 6, including for example microbes expected to counter the effects of acidosis), or (ii) a carrier configured to adsorb a solvent or to counter the effects of solventogenesis leading to acidosis. As examples, the carrier can be at least one of Zeolite, charcoal, activated granulated charcoal, bone ash, bone charcoal, diatomaceous earth, kaolin, and clay. In some implementations, an ADS can be used (implementing a health prediction and/or an animal management process described herein) to recommend a feed supplement to be provided to the animal, the feed supplement including a microbial growth inhibitor configured to inhibit the increase in microbial lead that eventually leads to solventogenesis and configured to induce a recovery from the state of acidosis. In some implementations, the microbial growth inhibitor can be at least one of ascorbic acid, benzoic acid, propionic acid, sodium sulfite, sulfur dioxide, sodium nitrite, and fumaric acid. In some embodiments, the microbial growth inhibitor can be combinations of the above listed ingredients, that is, combinations of adsorptive ingredients, combinations of microbial growth inhibitors, and/or combinations of adsorptive ingredient(s) and inhibitor(s). The method at 750 can include predicting a progression of a state of acidosis and/or a state of recovery given the recommended interventions and/or feed supplements. In some example embodiments, an example amount of additive to be included can be approximately 0.5 g-2 kg 268 g (5 lbs) per head per day for each non-microbe ingredient. In some embodiments, in addition to the additives one or more carriers can be added (e.g., the remaining volume can be filled with calcium carbonate).

Alternatively and/or additionally, the method at 730B, the method 700 includes determining a state of no acidosis based on the measurement being lesser than the predetermined baseline measurement. In some implementations, the method can include more than one predetermined baseline measurements to also determine a risk of onset of acidosis. For example, the method at 730B can include determining a state of no acidosis based on a test measurement of concentration of ethanol being lesser than a first predetermined baseline measurement but the test measurement being within range of a second predetermined positive control measurement associated with a stage associated with a statistically significant risk of onset of acidosis unless averted by positive action.

At 740B, the method includes generating a feed recommendation based on the determination of the state of no acidosis and/or a state of risk of onset of acidosis. And at 750B, the method includes predicting a state of health and/or prevention of a state of acidosis if following a recommended animal management plan including one or more feed supplements. As described herein, the feed supplements can include one or more of a synthetic microbial ensemble configure to counteract the biomass shift towards acidosis, a carrier configured to absorb a solvent associate with acidosis, and/or a microbial growth inhibitor configured to inhibit solventogenesis, as described above.

### Microbial Ensembles

In some embodiments, the disclosure provides microbial products produced by the methods described herein and comprising at least one microorganism, wherein the at least one microorganism is disclosed in one or more of the following: U.S. Pat. App. Pub. Nos. 2018/0310592, 2018/0333443, 2018/0223325, 2022/0174992, 2022/0265732, and PCT Pub. Nos. 2020/227442, 2021/163212, 2021/202804, 2022/226367, and 2022/081992, (each being herein expressly incorporated by reference for all purposes).

### Isolated Microbes—Source Material

The microbes of the present disclosure were obtained, among other places, at various locales in the United States from the gastrointestinal tract of cows.

### Isolated Microbes—Microbial Culture Techniques

The microbes of Tables 1, 2, 3, 4, 5, and/or 6 were matched to their nearest taxonomic groups by utilizing classification tools of the Ribosomal Database Project (RDP) for 16s IRNA sequences and the User-friendly Nordic ITS Ectomycorrhiza (UNITE) database for ITS rRNA sequences. Examples of matching microbes to their nearest taxa may be found in Lan et al. (2012. PLOS one. 7(3):e32491), Schloss and Westcott (2011. Appl. Environ. Microbiol. 77(10):3219-3226), and Koljalg et al. (2005. New Phytologist. 166(3):1063-1068).

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., positive/negative, Gram capable of forming spores aerobically/anaerobically, cellular morphology, carbon source metabolism, acid/base production, enzyme secretion, metabolic secretions, etc.) and to reduce the likelihood of working with a culture which has become contaminated.

For example, for microbes of the disclosure, biologically pure isolates can be obtained through repeated subculture of biological samples, each subculture followed by streaking onto solid media to obtain individual colonies or colony forming units. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze dried liquid formulations and cultures stored long term at −70° C. in solutions containing glycerol are contemplated for use in providing formulations of the present disclosure.

The microbes of the disclosure can be propagated in a liquid medium under aerobic conditions, or alternatively anaerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the microbes include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present disclosure include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the microbial strains, essentially, between 20° C. and 46° C. In some aspects, a temperature range is 30° C.-39° C. For optimal growth, in some embodiments, the medium can be adjusted to pH 6.0-7.4. It will be appreciated that commercially available media may also be used to culture the microbial strains, such as Nutrient Broth or Nutrient Agar available from Difco, Detroit, MI. It will be appreciated that cultivation time may differ depending on the type of culture medium used and the concentration of sugar as a major carbon source.

In some aspects, cultivation lasts between 24-96 hours. Microbial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. In some aspects, microbial multi-strain cultures may be obtained by propagating two or more of the strains described hereinabove. It will be appreciated that the microbial strains may be cultured together when compatible culture conditions can be employed.

In some embodiments, the microorganisms of the present disclosure are subjected to a serial preservation challenge to improve microbial viability. In some embodiments, the microorganisms are subjected to a serial preservation challenge to improve stability. In some embodiments, the microorganisms of the present disclosure are subjected to at least one preservation challenge. In some embodiments, the microorganisms of the present disclosure are subjected to at least two, three, four, five, or more preservation challenges.

In some embodiments, the serial preservation method comprises: (a) subjecting a population of target microbial cells to a first preservation challenge to provide a first population of challenged microbial cells; (b) harvesting viable challenged microbial cells from the first population of challenged microbial cells to provide a first population of viable challenged microbial cells; (c) subjecting the first population of viable challenged microbial cells to a second preservation challenge to provide a second population of challenged microbial cells; (d) harvesting viable challenged microbial cells from the second population of challenged microbial cells to provide a second population of viable challenged microbial cells; (e) subjecting the second population of viable challenged microbial cells to a third preservation challenge to provide a third population of challenged microbial cells; (f) harvesting viable challenged microbial cells from the third population of challenged microbial cells to provide a third population of viable challenged microbial cells; (g) preserving the third population of viable challenged microbial cells to provide a population of preserved viability-enhanced microbial cells; and (h) preparing a product using the population of preserved viability-enhanced microbial cells.

In some embodiments, each of the preservation challenges are the same type of preservation challenge. For example, in some embodiments, the microorganisms of the present disclosure are subjected to two, three, four, five, or more preservation challenges before final preservation for storage and/or incorporation into a product, wherein each of the preservation challenges are of the same type. Types of preservation challenges include, but are not limited to, freeze drying/lyophilization, vitrification/glass formation, evaporation, foam formation, vaporization, cryopreservation, spray drying, adsorptive drying, extrusion, and fluid bed drying. Methods of microbial preservation are further described in PCT Application No. PCT/US2020/020311, herein incorporated by reference in its entirety.

In some embodiments, the preservation challenges are different types of preservation challenges. For example, in some embodiments, the microorganisms of the present disclosure are subjected to a first and a second preservation challenge, wherein the first and the second preservation challenges are different challenges types. For example, in some embodiments, the first preservation challenge is a cryopreservation challenge and the second preservation challenge is a freeze-drying preservation challenge.

In some embodiments, any one of the microorganisms listed in Table 1, 2 or 3 may be subjected to serial preservation challenge. In some embodiments, a microorganism comprising a 16S nucleic acid sequence with at least 95% sequence identity to SEQ ID NOs: 1-30, 2045-2103, 2108, or 2125-4945 is subjected to serial preservation challenge. In some embodiments, a microorganism comprising an ITS nucleic acid sequence with at least 95% sequence identity to SEQ ID NOs: 31-60 and 2104-2107 is subjected to serial preservation challenge.

In some embodiments, serial preservation results in one or more mutations in the genome of a microorganism. In some embodiments, serial preservation results in one or more mutations in the genome of any one of the microorganisms listed in Table 1, 2 or 3. In some embodiments, serial preservation results in one or more mutations in a microorganism comprising a 16S nucleic acid sequence with at least 95% sequence identity to SEQ ID NOs: 1-30, 2045-2103, 2108, or 2125-4945. In some embodiments, serial preservation results in one or more mutations in a microorganism comprising an ITS nucleic acid sequence with at least 95% sequence identity to SEQ ID NOs: 31-60 and 2104-2107.

In some embodiments, serial preservation results in one or more mutations in Ruminococcus bovis comprising a 16S nucleic acid sequence of SEQ ID NO: 2108. In some embodiments, the one or more mutations are located in the whole genome of Ruminococcus bovis comprising a 16S nucleic acid sequence of SEQ ID NO: 2108. In some embodiments, the one or mutations are not located in the 16S nucleic acid sequence of SEQ ID NO: 2108 of Ruminococcus bovis.

### Isolated Microbes—Microbial Strains

Microbes can be distinguished into a genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTm (the difference in the melting temperature between homologous and heterologous hybrids), under standard conditions, are considered to be members of the same species. Thus, populations that share greater than the aforementioned 70% threshold can be considered to be variants of the same species. Another accepted genotypic method for defining species is to isolate marker genes of the present disclosure, sequence these genes, and align these sequenced genes from multiple isolates or variants. The microbes are interpreted as belonging to the same species if one or more of the sequenced genes share at least 97% sequence identity.

The 16S or 18S rRNA sequences or ITS sequences are often used for making distinctions between species and strains, in that if one of the aforementioned sequences share less than a specified percent sequence identity from a reference sequence, then the two organisms from which the sequences were obtained are said to be of different species or strains.

Thus, one could consider microbes to be of the same species, if they share at least 80%, 85%, 90%, 95%, 97%, 98%, or 99% sequence identity across the 16S or 18S rRNA sequence, or the ITS1 or ITS2 sequence.

Further, one could define microbial strains of a species, as those that share at least 80%, 85%, 90%, 95%, 97%, 98%, or 99% sequence identity across the 16S or 18S rRNA sequence, or the ITS1 or ITS2 sequence.

In one embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, and 2125-4945. In a further embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-4945.

In one embodiment, microbial strains of the present disclosure include those that comprise polynucleotide sequences that share at least 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% sequence identity with any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, and 2125-4945.

Comparisons may also be made with 23S rRNA sequences against reference sequences.

Unculturable microbes often cannot be assigned to a definite species in the absence of a phenotype determination, the microbes can be given a candidatus designation within a genus provided their 16S or 18S rRNA sequences or ITS sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics are made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity and atmospheric conditions, growth in presence of different sole carbon and nitrogen sources. One of ordinary skill in the art would be reasonably apprised as to the phenotypic traits that define the genera of the present disclosure.

In one embodiment, the microbes taught herein were identified utilizing 16S rRNA gene sequences and ITS sequences. It is known in the art that 16S rRNA contains hypervariable regions that can provide species/strain-specific signature sequences useful for bacterial identification, and that ITS sequences can also provide species/strain-specific signature sequences useful for fungal identification.

Phylogenetic analysis using the rRNA genes and/or ITS sequences are used to define “substantially similar” species belonging to common genera and also to define “substantially similar” strains of a given taxonomic species. Furthermore, physiological and/or biochemical properties of the isolates can be utilized to highlight both minor and significant differences between strains that could lead to advantageous behavior in ruminants.

Compositions of the present disclosure may include combinations of fungal spores and bacterial spores, fungal spores and bacterial vegetative cells, fungal vegetative cells and bacterial spores, fungal vegetative cells and bacterial vegetative cells. In some embodiments, compositions of the present disclosure comprise bacteria only in the form of spores. In some embodiments, compositions of the present disclosure comprise bacteria only in the form of vegetative cells. In some embodiments, compositions of the present disclosure comprise bacteria in the absence of fungi. In some embodiments, compositions of the present disclosure comprise fungi in the absence of bacteria.

Bacterial spores may include endospores and akinetes. Fungal spores may include statismospores, ballistospores, autospores, aplanospores, zoospores, mitospores, megaspores, microspores, meiospores, chlamydospores, urediniospores, teliospores, oospores, carpospores, tetraspores, sporangiospores, zygospores, ascospores, basidiospores, ascospores, and asciospores.

In some embodiments, spores of the composition germinate upon administration to animals of the present disclosure. In some embodiments, spores of the composition germinate only upon administration to animals of the present disclosure.

### Microbial Compositions

In some embodiments, the microbes of the disclosure are combined into microbial compositions.

In some embodiments, the microbial compositions include ruminant feed, such as cereals (barley, maize, oats, and the like); starches (tapioca and the like); oilseed cakes; and vegetable wastes. In some embodiments, the microbial compositions include vitamins, minerals, amino acids, enzymes, trace elements, emulsifiers, aromatizing products, binders, colorants, odorants, thickening agents, and the like. In some embodiments, the microbial compositions are combined with medicines or vaccines.

In some embodiments, the microbial compositions of the present disclosure are solid. Where solid compositions are used, it may be desired to include one or more carrier materials including, but not limited to: mineral earths such as silicas, talc, kaolin, limestone, chalk, clay, dolomite, diatomaceous earth; calcium sulfate; magnesium sulfate; magnesium oxide; products of vegetable origin such as cereal meals, tree bark meal, wood meal, and nutshell meal.

In some embodiments, the microbial compositions of the present disclosure are liquid. In further embodiments, the liquid comprises a solvent that may include water or an alcohol, and other animal-safe solvents. In some embodiments, the microbial compositions of the present disclosure include binders such as animal-safe polymers, carboxymethylcellulose, starch, polyvinyl alcohol, and the like.

In some embodiments, the microbial compositions of the present disclosure comprise thickening agents such as silica, clay, natural extracts of seeds or seaweed, synthetic derivatives of cellulose, guar gum, locust bean gum, alginates, and methylcelluloses. In some embodiments, the microbial compositions comprise anti-settling agents such as modified starches, polyvinyl alcohol, xanthan gum, and the like.

In some embodiments, the microbial compositions of the present disclosure comprise colorants including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. In some embodiments, the microbial compositions of the present disclosure comprise trace nutrients such as salts of iron, manganese, boron, copper, cobalt, molybdenum and zinc.

In some embodiments, the microbial compositions of the present disclosure comprise an animal-safe virucide or nematicide.

In some embodiments, microbial compositions of the present disclosure comprise saccharides (e.g., monosaccharides, disaccharides, trisaccharides, polysaccharides, oligosaccharides, and the like), polymeric saccharides, lipids, polymeric lipids, lipopolysaccharides, proteins, polymeric proteins, lipoproteins, nucleic acids, nucleic acid polymers, silica, inorganic salts and combinations thereof. In a further embodiment, microbial compositions comprise polymers of agar, agarose, gelrite, gellan gumand the like. In some embodiments, microbial compositions comprise plastic capsules, emulsions (e.g., water and oil), membranes, and artificial membranes. In some embodiments, emulsions or linked polymer solutions may comprise microbial compositions of the present disclosure. See Harel and Bennett (U.S. Pat. No. 8,460,726B2).

In some embodiments, microbial compositions of the present disclosure occur in a solid form (e.g., dispersed lyophilized spores) or a liquid form (microbes interspersed in a storage medium).

In some embodiments, microbial compositions of the present disclosure comprise one or more preservatives. The preservatives may be in liquid or gas formulations. The preservatives may be selected from one or more of monosaccharide, disaccharide, trisaccharide, polysaccharide, acetic acid, ascorbic acid, calcium ascorbate, erythorbic acid, iso-ascorbic acid, erythrobic acid, potassium nitrate, sodium ascorbate, sodium erythorbate, sodium iso-ascorbate, sodium nitrate, sodium nitrite, nitrogen, benzoic acid, calcium sorbate, ethyl lauroyl arginate, methyl-p-hydroxy benzoate, methyl paraben, potassium acetate, potassium benzoiate, potassium bisulphite, potassium diacetate, potassium lactate, potassium metabisulphite, potassium sorbate, propyl-p-hydroxy benzoate, propyl paraben, sodium acetate, sodium benzoate, sodium bisulphite, sodium nitrite, sodium diacetate, sodium lactate, sodium metabisulphite, sodium salt of methyl-p-hydroxy benzoic acid, sodium salt of propyl-p-hydroxy benzoic acid, sodium sulphate, sodium sulfite, sodium dithionite, sulphurous acid, calcium propionate, dimethyl dicarbonate, natamycin, potassium sorbate, potassium bisulfite, potassium metabisulfite, propionic acid, sodium diacetate, sodium propionate, sodium sorbate, sorbic acid, ascorbic acid, ascorbyl palmitate, ascorbyl stearate, butylated hydro-xyanisole, butylated hydroxytoluene (BHT), butylated hydroxyl anisole (BHA), citric acid, citric acid esters of mono-and/or diglycerides, L-cysteine, L-cysteine hydrochloride, gum guaiacum, gum guaiac, lecithin, lecithin citrate, monoglyceride citrate, monoisopropyl citrate, propyl gallate, sodium metabisulphite, tartaric acid, tertiary butyl hydroquinone, stannous chloride, thiodipropionic acid, dilauryl thiodipropionate, distearyl thiodipropionate, ethoxyquin, sulfur dioxide, formic acid, or tocopherol(s).

In some embodiments, microbial compositions of the present disclosure include bacterial and/or fungal cells in spore form, vegetative cell form, and/or lysed cell form. In one embodiment, the lysed cell form acts as a mycotoxin binder, e.g. mycotoxins binding to dead cells.

In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable in a refrigerator (35-40° F.) for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at room temperature (68-72° F.) or between 50-77° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at −23-35° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 77-100° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the microbial compositions are shelf stable at 101-213° F. for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weeks.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of about 1 to 100, about 1 to 95, about 1 to 90, about 1 to 85, about 1 to 80, about 1 to 75, about 1 to 70, about 1 to 65, about 1 to 60, about 1 to 55, about 1 to 50, about 1 to 45, about 1 to 40, about 1 to 35, about 1 to 30, about 1 to 25, about 1 to 20, about 1 to 15, about 1 to 10, about 1 to 5, about 5 to 100, about 5 to 95, about 5 to 90, about 5 to 85, about 5 to 80, about 5 to 75, about 5 to 70, about 5 to 65, about 5 to 60, about 5 to 55, about 5 to 50, about 5 to 45, about 5 to 40, about 5 to 35, about 5 to 30, about 5 to 25, about 5 to 20, about 5 to 15, about 5 to 10, about 10 to 100, about 10 to 95, about 10 to 90, about 10 to 85, about 10 to 80, about 10 to 75, about 10 to 70, about 10 to 65, about 10 to 60, about 10 to 55, about 10 to 50, about 10 to 45, about 10 to 40, about 10 to 35, about 10 to 30, about 10 to 25, about 10 to 20, about 10 to 15, about 15 to 100, about 15 to 95, about 15 to 90, about 15 to 85, about 15 to 80, about 15 to 75, about 15 to 70, about 15 to 65, about 15 to 60, about 15 to 55, about 15 to 50, about 15 to 45, about 15 to 40, about 15 to 35, about 15 to 30, about 15 to 25, about 15 to 20, about 20 to 100, about 20 to 95, about 20 to 90, about 20 to 85, about 20 to 80, about 20 to 75, about 20 to 70, about 20 to 65, about 20 to 60, about 20 to 55, about 20 to 50, about 20 to 45, about 20 to 40, about 20 to 35, about 20 to 30, about 20 to 25, about 25 to 100, about 25 to 95, about 25 to 90, about 25 to 85, about 25 to 80, about 25 to 75, about 25 to 70, about 25 to 65, about 25 to 60, about 25 to 55, about 25 to 50, about 25 to 45, about 25 to 40, about 25 to 35, about 25 to 30, about 30 to 100, about 30 to 95, about 30 to 90, about 30 to 85, about 30 to 80, about 30 to 75, about 30 to 70, about 30 to 65, about 30 to 60, about 30 to 55, about 30 to 50, about 30 to 45, about 30 to 40, about 30 to 35, about 35 to 100, about 35 to 95, about 35 to 90, about 35 to 85, about 35 to 80, about 35 to 75, about 35 to 70, about 35 to 65, about 35 to 60, about 35 to 55, about 35 to 50, about 35 to 45, about 35 to 40, about 40 to 100, about 40 to 95, about 40 to 90, about 40 to 85, about 40 to 80, about 40 to 75, about 40 to 70, about 40 to 65, about 40 to 60, about 40 to 55, about 40 to 50, about 40 to 45, about 45 to 100, about 45 to 95, about 45 to 90, about 45 to 85, about 45 to 80, about 45 to 75, about 45 to 70, about 45 to 65, about 45 to 60, about 45 to 55, about 45 to 50, about 50 to 100, about 50 to 95, about 50 to 90, about 50 to 85, about 50 to 80, about 50 to 75, about 50 to 70, about 50 to 65, about 50 to 60, about 50 to 55, about 55 to 100, about 55 to 95, about 55 to 90, about 55 to 85, about 55 to 80, about 55 to 75, about 55 to 70, about 55 to 65, about 55 to 60, about 60 to 100, about 60 to 95, about 60 to 90, about 60 to 85, about 60 to 80, about 60 to 75, about 60 to 70, about 60 to 65, about 65 to 100, about 65 to 95, about 65 to 90, about 65 to 85, about 65 to 80, about 65 to 75, about 65 to 70, about 70 to 100, about 70 to 95, about 70 to 90, about 70 to 85, about 70 to 80, about 70 to 75, about 75 to 100, about 75 to 95, about 75 to 90, about 75 to 85, about 75 to 80, about 80 to 100, about 80 to 95, about 80 to 90, about 80 to 85, about 85 to 100, about 85 to 95, about 85 to 90, about 90 to 100, about 90 to 95, or 95 to 100 weeks

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10, 1 to 5, 5 to 100, 5 to 95, 5 to 90, 5 to 85, to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, 5 to 10, 10 to 100, 10 to 95, 10 to 90, 10 to 85, 10 to 80, 10 to 75, 10 to 70, to 65, 10 to 60, 10 to 55, 10 to 50, 10 to 45, 10 to 40, 10 to 35, 10 to 30, 10 to 25, 10 to 20, 10 to 15, 15 to 100, 15 to 95, 15 to 90, 15 to 85, 15 to 80, 15 to 75, 15 to 70, 15 to 65, 15 to 60, to 55, 15 to 50, 15 to 45, 15 to 40, 15 to 35, 15 to 30, 15 to 25, 15 to 20, 20 to 100, 20 to 95, to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, to 70, 25 to 65, 25 to 60, 25 to 55, 25 to 50, 25 to 45, 25 to 40, 25 to 35, 25 to 30, 30 to 100, to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, to 65, 35 to 60, 35 to 55, 35 to 50, 35 to 45, 35 to 40, 40 to 100, 40 to 95, 40 to 90, 40 to 85, to 80, 40 to 75, 40 to 70, 40 to 65, 40 to 60, 40 to 55, 40 to 50, 40 to 45, 45 to 100, 45 to 95, to 90, 45 to 85, 45 to 80, 45 to 75, 45 to 70, 45 to 65, 45 to 60, 45 to 55, 45 to 50, 50 to 100, to 95, 50 to 90, 50 to 85, 50 to 80, 50 to 75, 50 to 70, 50 to 65, 50 to 60, 50 to 55, 55 to 100, to 95, 55 to 90, 55 to 85, 55 to 80, 55 to 75, 55 to 70, 55 to 65, 55 to 60, 60 to 100, 60 to 95, to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 weeks.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of about 1 to 36, about 1 to 34, about 1 to 32, about 1 to 30, about 1 to 28, about 1 to 26, about 1 to 24, about 1 to 22, about 1 to 20, about 1 to 18, about 1 to 16, about 1 to 14, about 1 to 12, about 1 to 10, about 1 to 8, about 1 to 6, about 1 one 4, about 1 to 2, about 4 to 36, about 4 to 34, about 4 to 32, about 4 to 30, about 4 to 28, about 4 to 26, about 4 to 24, about 4 to 22, about 4 to 20, about 4 to 18, about 4 to 16, about 4 to 14, about 4 to 12, about 4 to 10, about 4 to 8, about 4 to 6, about 6 to 36, about 6 to 34, about 6 to 32, about 6 to 30, about 6 to 28, about 6 to 26, about 6 to 24, about 6 to 22, about 6 to 20, about 6 to 18, about 6 to 16, about 6 to 14, about 6 to 12, about 6 to 10, about 6 to 8, about 8 to 36, about 8 to 34, about 8 to 32, about 8 to 30, about 8 to 28, about 8 to 26, about 8 to 24, about 8 to 22, about 8 to 20, about 8 to 18, about 8 to 16, about 8 to 14, about 8 to 12, about 8 to 10, about 10 to 36, about 10 to 34, about 10 to 32, about 10 to 30, about 10 to 28, about 10 to 26, about 10 to 24, about 10 to 22, about 10 to 20, about 10 to 18, about 10 to 16, about 10 to 14, about 10 to 12, about 12 to 36, about 12 to 34, about 12 to 32, about 12 to 30, about 12 to 28, about 12 to 26, about 12 to 24, about 12 to 22, about 12 to 20, about 12 to 18, about 12 to 16, about 12 to 14, about 14 to 36, about 14 to 34, about 14 to 32, about 14 to 30, about 14 to 28, about 14 to 26, about 14 to 24, about 14 to 22, about 14 to 20, about 14 to 18, about 14 to 16, about 16 to 36, about 16 to 34, about 16 to 32, about 16 to 30, about 16 to 28, about 16 to 26, about 16 to 24, about 16 to 22, about 16 to 20, about 16 to 18, about 18 to 36, about 18 to 34, about 18 to 32, about 18 to 30, about 18 to 28, about 18 to 26, about 18 to 24, about 18 to 22, about 18 to 20, about 20 to 36, about 20 to 34, about 20 to 32, about 20 to 30, about 20 to 28, about 20 to 26, about 20 to 24, about 20 to 22, about 22 to 36, about 22 to 34, about 22 to 32, about 22 to 30, about 22 to 28, about 22 to 26, about 22 to 24, about 24 to 36, about 24 to 34, about 24 to 32, about 24 to 30, about 24 to 28, about 24 to 26, about 26 to 36, about 26 to 34, about 26 to 32, about 26 to 30, about 26 to 28, about 28 to 36, about 28 to 34, about 28 to 32, about 28 to 30, about 30 to 36, about 30 to 34, about 30 to 32, about 32 to 36, about 32 to 34, or about 34 to 36 months.

In some embodiments, the microbial compositions of the present disclosure are shelf stable at refrigeration temperatures (35-40° F.), at room temperature (68-72° F.), between 50-77° F., between −23-35° F., between 70-100° F., or between 101-213° F. for a period of 1 to 36 1 to 34 1 to 32 1 to 30 1 to 28 1 to 26 1 to 24 1 to 22 1 to 20 1 to 18 1 to 16 1 to 14 1 to 12 1 to 10 1 to 8 1 to 6 1 one 4 1 to 2 4 to 36 4 to 34 4 to 32 4 to 30 4 to 28 4 to 26 4 to 24 4 to 22 4 to 20 4 to 18 4 to 16 4 to 14 4 to 12 4 to 10 4 to 8 4 to 6 6 to 36 6 to 34 6 to 32 6 to 30 6 to 28 6 to 26 6 to 24 6 to 22 6 to 20 6 to 18 6 to 16 6 to 14 6 to 12 6 to 10 6 to 8 8 to 36 8 to 34 8 to 32 8 to 30 8 to 28 8 to 26 8 to 24 8 to 22 8 to 20 8 to 18 8 to 16 8 to 14 8 to 12 8 to 10 10 to 36 10 to 34 10 to 32 10 to 30 10 to 28 10 to 26 10 to 24 10 to 22 10 to 20 10 to 18 10 to 16 10 to 14 10 to 12 12 to 36 12 to 34 12 to 32 12 to 30 12 to 28 12 to 26 12 to 24 12 to 22 12 to 20 12 to 18 12 to 16 12 to 14 14 to 36 14 to 34 14 to 32 14 to 30 14 to 28 14 to 26 14 to 24 14 to 22 14 to 20 14 to 18 14 to 16 16 to 36 16 to 34 16 to 32 16 to 30 16 to 28 16 to 26 16 to 24 16 to 22 16 to 20 16 to 18 18 to 36 18 to 34 18 to 32 18 to 30 18 to 28 18 to 26 18 to 24 18 to 22 18 to 20 20 to 36 20 to 34 20 to 32 20 to 30 20 to 28 20 to 26 20 to 24 20 to 22 22 to 36 22 to 34 22 to 32 22 to 30 22 to 28 22 to 26 22 to 24 24 to 36 24 to 34 24 to 32 24 to 30 24 to 28 24 to 26 26 to 36 26 to 34 26 to 32 26 to 30 26 to 28 28 to 36 28 to 34 28 to 32 28 to 30 30 to 36 30 to 34 30 to 32 32 to 36 32 to 34, or about 34 to 36.

In some embodiments, the microbial compositions of the present disclosure are shelf stable at any of the disclosed temperatures and/or temperature ranges and spans of time at a relative humidity of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, or 98%.

### Encapsulated Compositions

In some embodiments, the microbes or microbial compositions of the disclosure are encapsulated in an encapsulating composition. An encapsulating composition protects the microbes from external stressors prior to entering the gastrointestinal tract of ungulates. Encapsulating compositions further create an environment that may be beneficial to the microbes, such as minimizing the oxidative stresses of an aerobic environment on anaerobic microbes. See Kalsta et al. (U.S. Pat. No. 5,104,662A), Ford (U.S. Pat. No. 5,733,568A), and Mosbach and Nilsson (U.S. Pat. No. 4,647,536A) for encapsulation compositions of microbes, and methods of encapsulating microbes.

In one embodiment, the encapsulating composition comprises microcapsules having a multiplicity of liquid cores encapsulated in a solid shell material. For purposes of the disclosure, a “multiplicity” of cores is defined as two or more.

A first category of useful fusible shell materials is that of normally solid fats, including fats which are already of suitable hardness and animal or vegetable fats and oils which are hydrogenated until their melting points are sufficiently high to serve the purposes of the present disclosure. Depending on the desired process and storage temperatures and the specific material selected, a particular fat can be either a normally solid or normally liquid material. The terms “normally solid” and “normally liquid” as used herein refer to the state of a material at desired temperatures for storing the resulting microcapsules. Since fats and hydrogenated oils do not, strictly speaking, have melting points, the term “melting point” is used herein to describe the minimum temperature at which the fusible material becomes sufficiently softened or liquid to be successfully emulsified and spray cooled, thus roughly corresponding to the maximum temperature at which the shell material has sufficient integrity to prevent release of the choline cores. “Melting point” is similarly defined herein for other materials which do not have a sharp melting point.

Specific examples of fats and oils useful herein (some of which require hardening) are as follows: animal oils and fats, such as beef tallow, mutton tallow, lamb tallow, lard or pork fat, fish oil, and sperm oil; vegetable oils, such as canola oil, cottonseed oil, peanut oil, corn oil, olive oil, soybean oil, sunflower oil, safflower oil, coconut oil, palm oil, linseed oil, tung oil, and castor oil; fatty acid monoglycerides and diglycerides; free fatty acids, such as stearic acid, palmitic acid, and oleic acid; and mixtures thereof. The above listing of oils and fats is not meant to be exhaustive, but only exemplary.

Specific examples of fatty acids include linoleic acid, y-linoleic acid, dihomo-y-linolenic acid, arachidonic acid, docosatetraenoic acid, vaccenic acid, nervonic acid, mead acid, erucic acid, gondoic acid, elaidic acid, oleic acid, palitoleic acid, stearidonic acid, eicosapentaenoic acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecyclic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

Another category of fusible materials useful as encapsulating shell materials is that of waxes. Representative waxes contemplated for use herein are as follows: animal waxes, such as beeswax, lanolin, shell wax, and Chinese insect wax; vegetable waxes, such as carnauba, candelilla, bayberry, and sugar cane; mineral waxes, such as paraffin, microcrystalline petroleum, ozocerite, ceresin, and montan; synthetic waxes, such as low molecular weight polyolefin (e.g., CARBOWAX), and polyol ether-esters (e.g., sorbitol); Fischer-Tropsch process synthetic waxes; and mixtures thereof. Water-soluble waxes, such as CARBOWAX and sorbitol, are not contemplated herein if the core is aqueous.

Still other fusible compounds useful herein are fusible natural resins, such as rosin, balsam, shellac, and mixtures thereof.

Various adjunct materials are contemplated for incorporation in fusible materials according to the present disclosure. For example, antioxidants, light stabilizers, dyes and lakes, flavors, essential oils, anti-caking agents, fillers, pH stabilizers, sugars (monosaccharides, disaccharides, trisaccharides, and polysaccharides) and the like can be incorporated in the fusible material in amounts which do not diminish its utility for the present disclosure.

The core material contemplated herein constitutes from about 0.1% to about 50%, about 1% to about 35%. or about 5% to about 30% by weight of the microcapsules. In some embodiments, the core material contemplated herein constitutes no more than about 30% by weight of the microcapsules. In some embodiments, the core material contemplated herein constitutes about 5% by weight of the microcapsules. The core material is contemplated as either a liquid or solid at contemplated storage temperatures of the microcapsules.

The cores may include other additives well-known in the pharmaceutical art, including edible sugars, such as sucrose, glucose, maltose, fructose, lactose, cellobiose, monosaccharides, disaccharides, trisaccharides, polysaccharides, and mixtures thereof; artificial sweeteners, such as aspartame, saccharin, cyclamate salts, and mixtures thereof; edible acids, such as acetic acid (vinegar), citric acid, ascorbic acid, tartaric acid, and mixtures thereof; edible starches, such as corn starch; hydrolyzed vegetable protein; water-soluble vitamins, such as Vitamin C; water-soluble medicaments; water-soluble nutritional materials, such as ferrous sulfate; flavors; salts; monosodium glutamate; antimicrobial agents, such as sorbic acid; antimycotic agents, such as potassium sorbate, sorbic acid, sodium benzoate, and benzoic acid; food grade pigments and dyes; and mixtures thereof. Other potentially useful supplemental core materials will be apparent to those of ordinary skill in the art.

Emulsifying agents may be employed to assist in the formation of stable emulsions. Representative emulsifying agents include glyceryl monostearate, polysorbate esters, ethoxylated mono-and diglycerides, and mixtures thereof.

For ease of processing, and particularly to enable the successful formation of a reasonably stable emulsion, the viscosities of the core material and the shell material should be similar at the temperature at which the emulsion is formed. In particular, the ratio of the viscosity of the shell to the viscosity of the core, expressed in centipoise or comparable units, and both measured at the temperature of the emulsion, should be from about 22:1 to about 1:1, desirably from about 8:1 to about 1:1, and preferably from about 3:1 to about 1:1. A ratio of 1:1 would be ideal, but a viscosity ratio within the recited ranges is useful.

Encapsulating compositions are not limited to microcapsule compositions as disclosed above. In some embodiments encapsulating compositions encapsulate the microbial compositions in an adhesive polymer that can be natural or synthetic without toxic effect. In some embodiments, the encapsulating composition may be a matrix selected from sugar matrix, gelatin matrix, polymer matrix, silica matrix, starch matrix, foam matrix, etc. In some embodiments, the encapsulating composition may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; monosaccharides; fats; fatty acids, including oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

In some embodiments, the encapsulating shell of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

### Animal Feed

In some embodiments, compositions of the present disclosure are mixed with animal feed. In some embodiments, animal feed may be present in various forms such as pellets, capsules, granulated, powdered, liquid, or semi-liquid.

In some embodiments, compositions of the present disclosure are mixed into the premix at the feed mill (e.g., Carghill or Western Millin), alone as a standalone premix, and/or alongside other feed additives such as MONENSIN, vitamins, etc. In one embodiment, the compositions of the present disclosure are mixed into the feed at the feed mill. In another embodiment, compositions of the present disclosure are mixed into the feed itself.

In some embodiments, feed of the present disclosure may be supplemented with water, premix or premixes, forage, fodder, beans (e.g., whole, cracked, or ground), grains (e.g., whole, cracked, or ground), bean- or grain-based oils, bean-or grain-based meals, bean- or grain-based haylage or silage, bean-or grain-based syrups, fatty acids, sugar alcohols (e.g., polyhydric alcohols), commercially available formula feeds, and mixtures thereof.

In some embodiments, forage encompasses hay, haylage, and silage. In some embodiments, hays include grass hays (e.g., sudangrass, orchardgrass, or the like), alfalfa hay, and clover hay. In some embodiments, haylages include grass haylages, sorghum haylage, and alfalfa haylage. In some embodiments, silages include maize, oat, wheat, alfalfa, clover, and the like.

In some embodiments, premix or premixes may be utilized in the feed. Premixes may comprise micro-ingredients such as vitamins, minerals, amino acids; chemical preservatives; pharmaceutical compositions such as antibiotics and other medicaments; fermentation products, and other ingredients. In some embodiments, premixes are blended into the feed.

In some embodiments, the feed may include feed concentrates such as soybean hulls, sugar beet pulp, molasses, high protein soybean meal, ground corn, shelled corn, wheat midds, distiller grain, cottonseed hulls, rumen-bypass protein, rumen-bypass fat, and grease. See Luhman (U.S. Publication US20150216817A1), Anderson et al. (U.S. Pat. No. 3,484,243) and Porter and Luhman (U.S. Pat. No. 9,179,694B2) for animal feed and animal feed supplements capable of use in the present compositions and methods.

In some embodiments, feed occurs as a compound, which includes, in a mixed composition capable of meeting the basic dietary needs, the feed itself, vitamins, minerals, amino acids, and other necessary components. Compound feed may further comprise premixes.

In some embodiments, microbial compositions of the present disclosure may be mixed with animal feed, premix, and/or compound feed. Individual components of the animal feed may be mixed with the microbial compositions prior to feeding to ruminants. The microbial compositions of the present disclosure may be applied into or on a premix, into or on a feed, and/or into or on a compound feed.

### Administration of Microbial Compositions

In some embodiments, the microbial compositions of the present disclosure are administered to ruminants. In some embodiments, the microbial compositions of the present disclosure are administered to cattle such as steers, bulls, cows, heifers, or calves. In some embodiments, the microbial compositions of the present disclosure are administered to cows. In some embodiments, the microbial compositions of the present disclosure are administered to calves.

In some embodiments, the microbial compositions of the present disclosure are administered to ruminants via the oral route. In some embodiments the microbial compositions are administered via a direct injection route into the gastrointestinal tract. In further embodiments, the direct injection administration delivers the microbial compositions directly to the rumen. In some embodiments, the microbial compositions of the present disclosure are administered to animals anally. In further embodiments, anal administration is in the form of an inserted suppository.

In some embodiments, the microbial composition is administered in a dose comprise a total of, or at least, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41mL, 42 mL, 43 mL, 44 mL, 45 mL, 46 mL, 47 mL, 48 mL, 49 mL, 50 mL, 60 mL, 70 mL, 80 mL, 90 mL, 100 mL, 200 mL, 300 mL, 400 mL, 500 mL, 600 mL, 700 mL, 800 mL, 900 mL, or 1,000 mL.

In some embodiments, the microbial composition is administered in a dose comprising a total of, or at least, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶10⁵, 10⁴, 10³, or 10² microbial cells.

In some embodiments, the microbial compositions are mixed with feed, and the administration occurs through the ingestion of the microbial compositions along with the feed. In some embodiments, the dose of the microbial composition is administered such that there exists 10² to 10¹², 10³to 10¹², 10⁴ to 10¹², 10⁵ to 10¹², 10₆ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total microbial cells per gram or milliliter of the composition.

In some embodiments, the microbial compositions are mixed with feed, and the administration occurs through the ingestion of the microbial compositions along with the feed. In some embodiments, the dose of the microbial composition is administered such that there exists 10² to 10¹², 10³to 10¹², 10⁴ to 10¹², 10⁵ to 10¹², 10₆ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 10⁸, 10⁶ to 10⁸, 10⁷ to 10⁸, 10² to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² colony forming units per gram or milliliter of the composition.

In some embodiments, the administered dose of the microbial composition comprises 10² to 10¹⁸, 10³ to 10¹⁸, 10⁴ to 10¹⁸, 10⁵ to 10¹⁸, 10⁶ to 10¹⁸, 10⁷ to 10¹⁸, 10⁸ to 10¹⁸, 10⁹ to 10¹⁸, 10¹⁰ to 10¹⁸, 10¹¹ to 10¹⁸, 10¹² to 10¹⁸, 10¹³ to 10¹⁸, 10¹⁴ to 10¹⁸, 10¹⁵ to 10¹⁸, 10¹⁶ to 10¹⁸, 10¹⁷ to 10¹⁸, 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to b 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10³ to 10⁸, 10⁴ to 10⁸, 10⁵ to 108, 10⁶ to 10⁸, 10⁷ to 10⁸, 10²to 10⁷, 10³ to 10⁷, 10⁴ to 10⁷, 10⁵ to 10⁷, 10⁶ to 10⁷, 10² to 10⁶, 10³ to 10⁶, 10⁴ to 10⁶, 10⁵ to 10⁶, 10² to 10⁵, 10³ to 10⁵, 10⁴ to 10⁵, 10² to 10⁴, 10³ to 10⁴, 10² to 10³, 10¹⁸, 10¹⁷, 10¹⁶, 10¹⁵, 10¹⁴, 10¹³, 10¹², 10¹¹, 10¹⁰, 10⁹, 10⁸, 10⁷, 10⁶, 10⁵, 10⁴, 10³, or 10² total microbial cells.

In some embodiments, the administered dose of each microbe in the microbial composition is at least about, at least about 10³ colony forming units (CFU), at least about 10⁴ CFU, at least about 10⁵ CFU, at least about 10⁶ CFU, at least about 107 CFU, at least about 10⁸ CFU, at least about 10⁹ CFU, at least about 10¹⁰ CFU, at least about 10¹¹ CFU, at least about 10¹² CFU, at least about 10¹³ CFU, at least about 10¹⁴ CFU, at least about 10¹⁵ CFU, at least about 10¹⁶ CFU, at least about 10¹⁷ CFU, at least about 10¹⁸ CFU, at least about 10¹⁹ CFU, or at least about 10²⁰ CFU.

In some embodiments, the composition is administered 1 or more times per day. In some aspects, the composition is administered with food each time the animal is fed. In some embodiments, the composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 times per day.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 times per week.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 times per month.

In some embodiments, the microbial composition is administered 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 times per year.

In some embodiments, the feed can be uniformly coated with one or more layers of the microbes and/or microbial compositions disclosed herein, using conventional methods of mixing, spraying, or a combination thereof through the use of treatment application equipment that is specifically designed and manufactured to accurately, safely, and efficiently apply coatings. Such equipment uses various types of coating technology such as rotary coaters, drum coaters, fluidized bed techniques, spouted beds, rotary mists, or a combination thereof. Liquid treatments such as those of the present disclosure can be applied via either a spinning “atomizer” disk or a spray nozzle, which evenly distributes the microbial composition onto the feed as it moves though the spray pattern. In some aspects, the feed is then mixed or tumbled for an additional period of time to achieve additional treatment distribution and drying.

In some embodiments, the feed coats of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some embodiments, the microbial cells can be coated freely onto any number of compositions or they can be formulated in a liquid or solid composition before being coated onto a composition. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, it is contemplated that the solid or liquid microbial compositions of the present disclosure further contain functional agents e.g., activated carbon, minerals, vitamins, and other agents capable of improving the quality of the products or a combination thereof.

Methods of coating and compositions in use of said methods that are known in the art can be particularly useful when they are modified by the addition of one of the embodiments of the present disclosure. Such coating methods and apparatus for their application are disclosed in, for example: U.S. Pat. Nos. 8,097,245, and 7,998,502; and PCT Pat. App. Publication Nos. WO 2008/076975, WO 2010/138522, WO 2011/094469, WO 2010/111347, and WO 2010/111565 each of which is incorporated by reference herein.

In some embodiments, the microbes or microbial consortia of the present disclosure exhibit a synergistic effect, on one or more of the traits described herein, in the presence of one or more of the microbes or consortia coming into contact with one another. The synergistic effect obtained by the taught methods can be quantified, for example, according to Colby's formula (i.e., (E)=X+Y−(X*Y/100)). See Colby, R. S., “Calculating Synergistic and Antagonistic Responses of Herbicide Combinations,” 1967. Weeds. Vol. 15, pp. 20-22, incorporated herein by reference in its entirety. Thus, “synergistic” is intended to reflect an outcome/parameter/effect that has been increased by more than an additive amount.

In some embodiments, the microbes or microbial consortia of the present disclosure may be administered via bolus. In one embodiment, a bolus (e.g., capsule containing the composition) is inserted into a bolus gun, and the bolus gun is inserted into the buccal cavity and/or esophagas of the animal, followed by the release/injection of the bolus into the animal's digestive tract. In one embodiment, the bolus gun/applicator is a BOVIKALC bolus gun/applicator. In another embodiment, the bolus gun/applicator is a QUADRICAL gun/applicator.

In some embodiments, the microbes or microbial consortia of the present disclosure may be administered via drench. In one embodiment, the drench is an oral drench. A drench administration comprises utilizing a drench kit/applicator/syringe that injects/releases a liquid comprising the microbes or microbial consortia into the buccal cavity and/or esophagas of the animal.

In some embodiments, the microbes or microbial consortia of the present disclosure may be administered in a time-released fashion. The composition may be coated in a chemical composition, or may be contained in a mechanical device or capsule that releases the microbes or microbial consortia over a period of time instead all at once. In one embodiment, the microbes or microbial consortia are administered to an animal in a time-release capsule. In one embodiment, the composition may be coated in a chemical composition, or may be contained in a mechanical device or capsul that releases the mcirobes or microbial consortia all at once a period of time hours post ingestion.

In some embodiments, the microbes or microbial consortia are administered in a time-released fashion between 1 to 5, 1 to 10, 1 to 15, 1 to 20, 1 to 24, 1 to 25, 1 to 30, 1 to 35, 1 to 40, 1 to 45, 1 to 50, 1 to 55, 1 to 60, 1 to 65, 1 to 70, 1 to 75, 1 to 80, 1 to 85, 1 to 90, 1 to 95, or 1 to 100 hours.

In some embodiments, the microbes or microbial consortia are administered in a time-released fashion between 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 29, or 1 to 30 days.

### Microorganisms

As used herein the term “microorganism” should be taken broadly. It includes, but is not limited to, the two prokaryotic domains, Bacteria and Archaea, as well as eukaryotic fungi, protists, and viruses.

By way of example, the microorganisms may include species of the genera of: Clostridium, Ruminococcus, Roseburia, Hydrogenoanaerobacterium, Saccharofermentans, Papillibacter, Pelotomaculum, Butyricicoccus, Tannerella, Prevotella, Butyricimonas, Piromyces, Pichia, Candida, Vrystaatia, Orpinomyces, Neocallimastix, and Phyllosticta. The microorganisms may further include species belonging to the family of Lachnospiraceae, and the order of Saccharomycesles. In some embodiments, the microorganisms may include species of any genera disclosed herein.

In certain embodiments, the microorganism is unculturable. This should be taken to mean that the microorganism is not known to be culturable or is difficult to culture using methods known to one skilled in the art.

In one embodiment, the microbes are obtained from animals (e.g., mammals, reptiles, birds, and the like), soil (e.g., rhizosphere), air, water (e.g., marine, freshwater, wastewater sludge), sediment, oil, plants (e.g., roots, leaves, stems), agricultural products, and extreme environments (e.g., acid mine drainage or hydrothermal systems). In a further embodiment, microbes obtained from marine or freshwater environments such as an ocean, river, or lake. In a further embodiment, the microbes can be from the surface of the body of water, or any depth of the body of water (e.g., a deep sea sample).

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either administered to the GI tract of an ungulate, or concentrated by filtering or centrifugation, diluted to an appropriate concentration and administered to the GI tract of an ungulate with the bulk of the salt removed. By way of further example, microorganisms from mineralized or toxic sources may be similarly treated to recover the microbes for application to the ungulate to minimize the potential for damage to the animal.

In another embodiment, the microorganisms are used in a crude form, in which they are not isolated from the source material in which they naturally reside. For example, the microorganisms are provided in combination with the source material in which they reside; for example, fecal matter, cud, or other composition found in the gastrointestinal tract. In this embodiment, the source material may include one or more species of microorganisms.

In some embodiments, a mixed population of microorganisms is used in the methods of the disclosure.

In embodiments of the disclosure where the microorganisms are isolated from a source material (for example, the material in which they naturally reside), any one or a combination of a number of standard techniques which will be readily known to skilled persons may be used. However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

Whilst not essential, in one embodiment, the material containing the microorganisms may be pre-treated prior to the isolation process in order to either multiply all microorganisms in the material. Microorganisms can then be isolated from the enriched materials as disclosed above.

In certain embodiments, as mentioned herein before, the microorganism(s) may be used in crude form and need not be isolated from an animal or a media. For example, cud, feces, or growth media which includes the microorganisms identified to be of benefit to increased milk production in ungulates may be obtained and used as a crude source of microorganisms for the next round of the method or as a crude source of microorganisms at the conclusion of the method. For example, fresh feces could be obtained and optionally processed.

### Microbiome Shift and Abundance of Microbes

In some embodiments, the microbiome of a ruminant, including the rumen microbiome, comprises a diverse arrive of microbes with a wide variety of metabolic capabilities. The microbiome is influenced by a range of factors including diet, variations in animal metabolism, and breed, among others. Most bovine diets are plant-based and rich in complex polysaccharides that enrich the gastrointestinal microbial community for microbes capable of breaking down specific polymeric components in the diet. The end products of primary degradation sustains a chain of microbes that ultimately produce a range of organic acids together with hydrogen and carbon dioxide. Because of the complex and interlinked nature of the microbiome, changing the diet and thus substrates for primary degradation may have a cascading effect on rumen microbial metabolism, with changes in both the organic acid profiles and the methane levels produced, thus impacting the quality and quantity of animal production and or the products produced by the animal. See Menezes et al. (2011. FEMS Microbiol. Ecol. 78(2):256-265.)

In some aspects, the present disclosure is drawn to administering microbial compositions described herein to modulate or shift the microbiome of a ruminant.

In some embodiments, the microbiome is shifted through the administration of one or more microbes to the gastrointestinal tract. In further embodiments, the one or more microbes are those selected from Table 1, 3 or 14. In some embodiments, the microbiome shift or modulation includes a decrease or loss of specific microbes that were present prior to the administration of one or more microbes of the present disclosure. In some embodiments, the microbiome shift or modulation includes an increase in microbes that were present prior to the administration of one or more microbes of the present disclosure. In some embodiments, the microbiome shift or modulation includes a gain of one or more microbes that were not present prior to the administration of one or more microbes of the present disclosure. In a further embodiment, the gain of one or more microbes is a microbe that was not specifically included in the administered microbial consortium.

In some embodiments, the administration of microbes of the present disclosure results in a sustained modulation of the microbiome such that the administered microbes are present in the microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, or 10 days.

In some embodiments, the administration of microbes of the present disclosure results in a sustained modulation of the microbiome such that the administered microbes are present in the microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5,

In some embodiments, the administration of microbes of the present disclosure results in a sustained modulation of the microbiome such that the administered microbes are present in the microbiome for a period of at least 1 to 10, 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8,8 to 10, 8 to 9, 9 to 10, 1, 2, 3, 4, 5, 6, 7, 8, 9, 10, 11, or 12 months.

In some embodiments, the presence of the administered microbes are detected by sampling the gastrointestinal tract and using primers to amplify the 16S or 18S rDNA sequences, or the ITS rDNA sequences of the administered microbes. In some embodiments, the administered microbes are one or more of those selected from Table 1, 3 or 14, and the corresponding rDNA sequences are those selected from SEQ ID NOs: 1-60, SEQ ID NOs: 2045-2108 and the SEQ ID NOs identified in Table 3. In some embodiments, the administered microbes are any one of SEQ ID NOs: 2125-4945.

In some embodiments, the microbiome of a ruminant is measured by amplifying polynucleotides collected from gastrointestinal samples, wherein the polynucleotides may be 16S or 18S rDNA fragments, or ITS rDNA fragments of microbial rDNA. In one embodiment, the microbiome is fingerprinted by a method of denaturing gradient gel electrophoresis (DGGE) wherein the amplified rDNA fragments are sorted by where they denature, and form a unique banding pattern in a gel that may be used for comparing the microbiome of the same ruminant over time or the microbiomes of multiple ruminants. In another embodiment, the microbiome is fingerprinted by a method of terminal restriction fragment length polymorphism (T-RFLP), wherein labelled PCR fragments are digested using a restriction enzyme and then sorted by size. In a further embodiment, the data collected from the T-RFLP method is evaluated by nonmetric multidimensional scaling (nMDS) ordination and PERMANOVA statistics identify differences in microbiomes, thus allowing for the identification and measurement of shifts in the microbiome. See also Shanks et al. (2011. Appl. Environ. Microbiol. 77(9):2992-3001), Petri et al. (2013. PLOS one. 8(12):e83424), and Menezes et al. (2011. FEMS Microbiol. Ecol. 78(2):256-265.)

In some embodiments, the administration of microbes of the present disclosure results in a modulation or shift of the microbiome which further results in a desired phenotype or improved trait.

### Experiment I: Effects of Acidosis

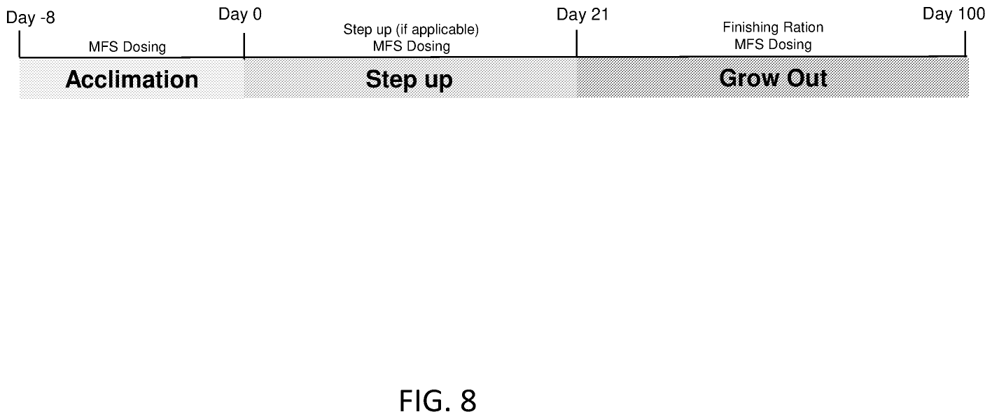

FIG. 8 illustrates a trial schedule associate with an example experiment designed to study effects associated with acidosis, using an ADS according to an embodiment.

Experiment I was designed to be a pen study with 2×2 factorial design, using 200steers, 10 steers per pen. Of the animals, 50 were designated to form the control group that had no step up including microbial feed supplement (MFS) dosing, and 50 were designated to form the treatment group, that had no step up and were supplemented with CO₂ utilizing rumen microorganisms, 50 were designated to form a control group that had a step up phase, and finally a 50 were designated to form a treatment group that experienced a step up phase where the animals were also supplemented with CO₂ utilizing rumen microorganisms.

As shown by the schedule in FIG. 8, the trial lasted 109 days with an acclimation period of 8 days, MFS were administered for the treatment groups prior to diet change in the step up period (the step-up period being either 0 days for the control groups or 21 days for the treatment groups), followed by a grow-out period that lasted 101 for the control groups or 80 days for the treatment groups. The product was mixed into feed daily, animals were fed ad-libitum.

Rumen measurements were taken including measurement of pH, dCO₂ (dissolved CO₂). VFAs, and Microbiome composition. Physiological measurements including body weight (˜monthly), average daily gain (ADG) which is a measurement of daily body weight change in animal on a feed test, weigh backs, and feed conversion ratio (FCR).

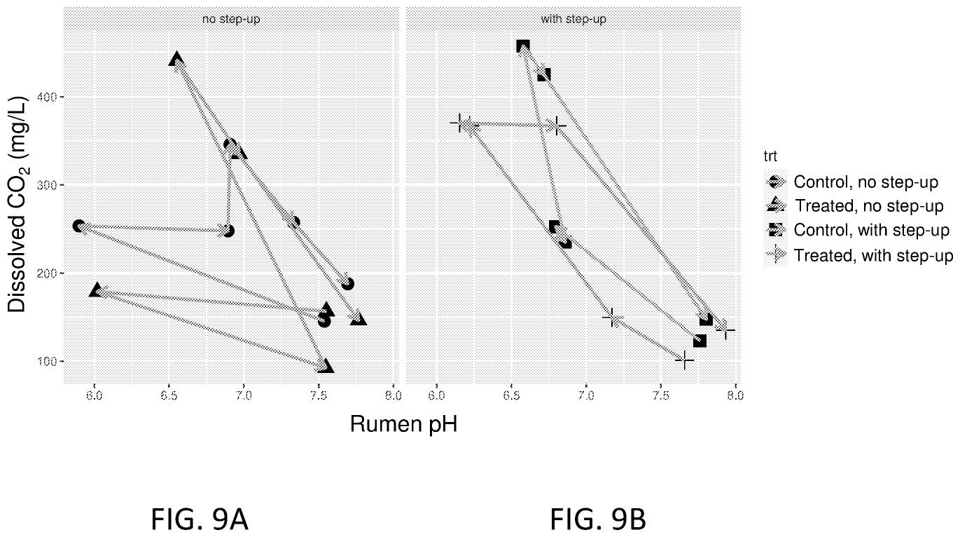

The results indicated that the step-up process allows the rumen microbiome to adapt and balance the production of VFAs, CO₂, and solvents. In the presence of alternative electron disposing pathway (anaerobic acetogenesis), CO₂ acted as an important electron sink. FIGS. 9A and 9B show plots of dissolved CO₂ as a function of rumen pH measured in the rumen. The measurements obtained from the treatment groups that underwent the step up phase showed an elevated amount of dissolved CO2 that was associated with relatively higher (less acidic) pH (indicating a reduced accumulation of VFAs) compared to the control groups that did not undergo the step up phase. Animals with the step up also showed a more gradual shift in pH and CO₂ over time, while the animals without the step up tended to have more severe spikes and lower pH values.

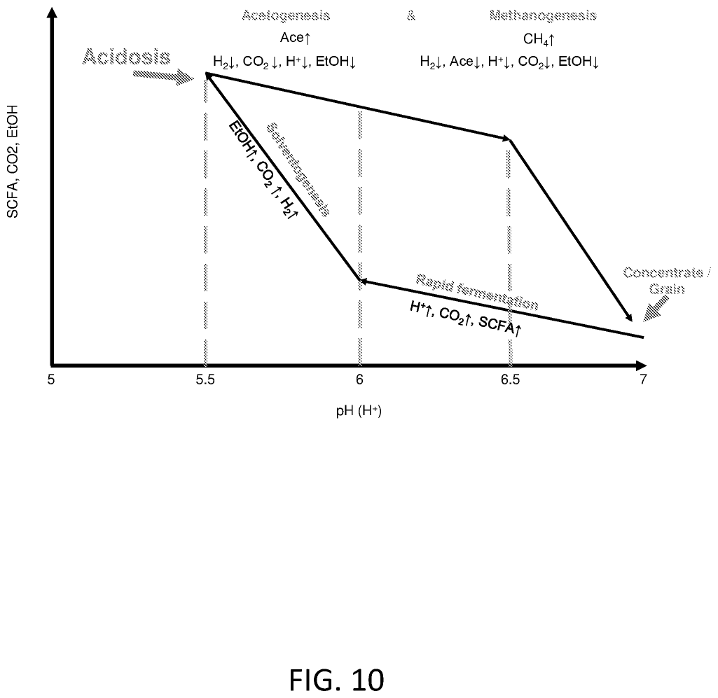

FIG. 10 shows a plot that illustrates a progression of acidosis in rumen of ruminants on diets that are extremely fermentable, for example finishing diets. Changes in microbial biomass resulting from highly fermentable diets is hypothesized to induce a unique microbial oscillation based on the differences in nutrient availability. A balance of microbial life in a biome depends on pathways for electron exchange. In the rumen, acidosis can be interpreted as an inability of the rumen to continue the cycle and regenerate its electron pool. In the absence of fiber and forages, a new oscillation forms between acidogenesis, solventogenesis, acetogenesis, and methanogenesis to regenerate electron pools. This hypothesis redefines acidosis as a consequence of disrupted electron exchange rather than a consequence of accumulation of concentration of lactate, a low pH, or the concentration of dCO2 which can all be correlated with and consequences of an interrupted cycle of electron exchange.

### Experiment II: Solvent Detection

This experiment was designed to test detection of solvents in saliva under baseline conditions and under conditions of induced acidosis challenge.

In a first phase of the experiment, the goal was to establish a predetermined baseline concentration of ethanol in a saliva of normal, lactating dairy cow over 1 day.

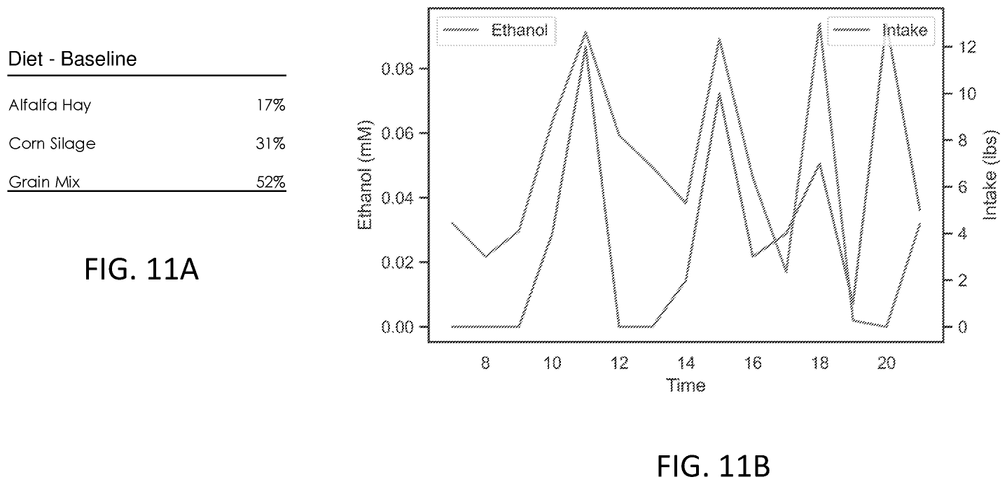

The study used one mid lactation dairy cow (days in milk DIM˜200), consuming standard farm total mixed ration (TMR) the constituents of which are shown in table form in FIG. 11A.

Data collection in the experiment included collecting saliva and blood serum hourly from the animal for ethanol measurements using bench test methods, an hourly measure of dry matter intake (DMI—an amount of feed a cow consumes per day on a moisture-free basis), and an hourly measurement of ethanol using an ADS using systems and/or methods described herein Saliva strip measurement.

FIG. 11B illustrates the predetermined measurement of baseline concentration of ethanol present in the saliva of a healthy lactating cow using bench test methods (potentially due to digestive products from ingesting silage, etc.). As shown the predetermined measurement of baseline concentration of ethanol in the saliva of a healthy cow consuming a typical TMR ration is between 0.00 mM to 0.1 mM over the course of an average day fluctuating based on feeding times of the day as shown by the correspondence of changes in ethanol concentration with changes in intake). Testing measurements from saliva showed test measurements were below noticeable threshold (i.e., indicated no detectable color change). There also was no ethanol detected in blood samples over the course of the day.

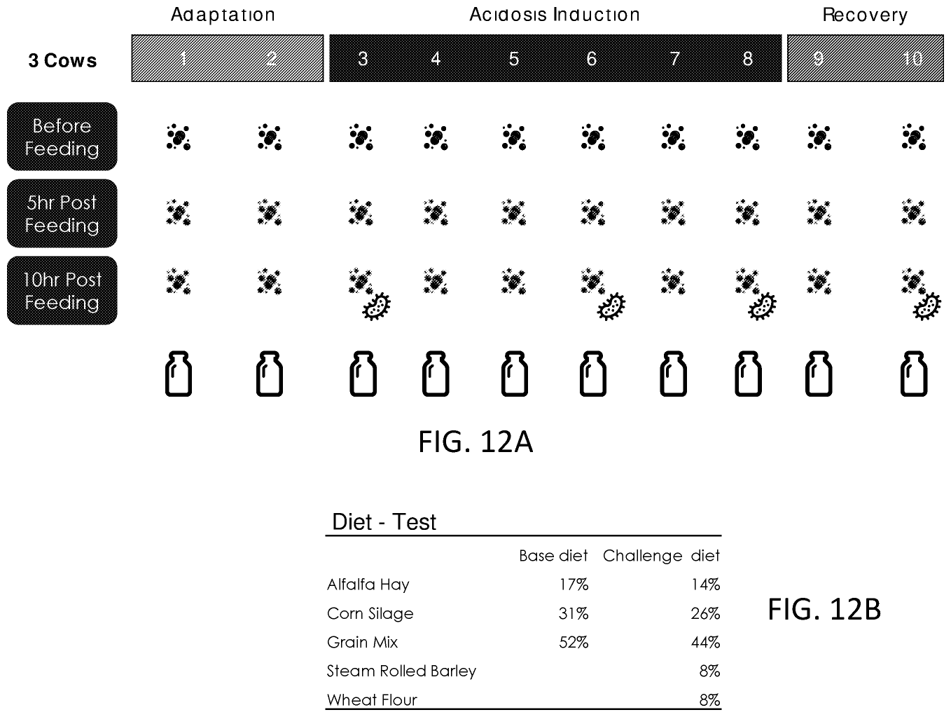

FIG. 12A is a schematic illustration of a schedule for induction of acidosis through an acidotic challenge protocol in a second phase of Experiment II. Determine saliva and ruminal ethanol concentrations in dairy cows undergoing acidosis/milk fat depression.

The study used three mid lactation dairy cows (days in milk (DIM) ˜200), consuming standard farm total mixed ration (see FIG. 11A). The animals were put on a schedule illustrated in FIG. 12A. As shown, days 1 and 2 were allocated for adaptation, days 3-8 were allocated for acidosis induction, and days 9 and 10 were allocated for recovery.

On day 1, the cows were switched to a test diet the constituents of which are shown in tabulated form in FIG. 12B. Data collection in the experiment included collecting milk yield and milk components such as milk fat, milk protein, (daily), collecting measure of dry matter intake DMI (daily), collecting rumen fluid for pH and microbiome analysis on day 2 (adaptation), on days 5 and 8 (acidosis challenge day 3 and 6, respectively), and day 10 (recovery day 2), collecting saliva and blood serum samples for ethanol measurements (3× per day, before feeding, 5 hr post feeding, and 10 hr. post feeding, as shown in the schedule in FIG. 12A), collecting sample of saliva for examination and ethanol detection and measurement using an ADS using systems and/or method described herein, and collecting sample of diet sampling for analysis.

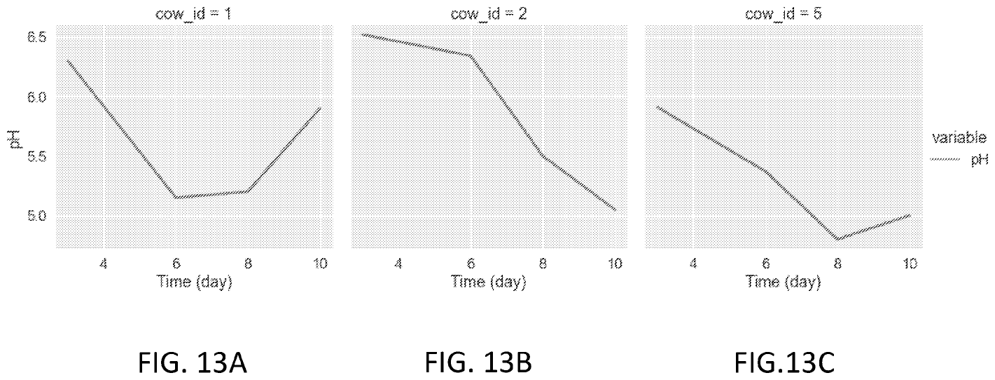

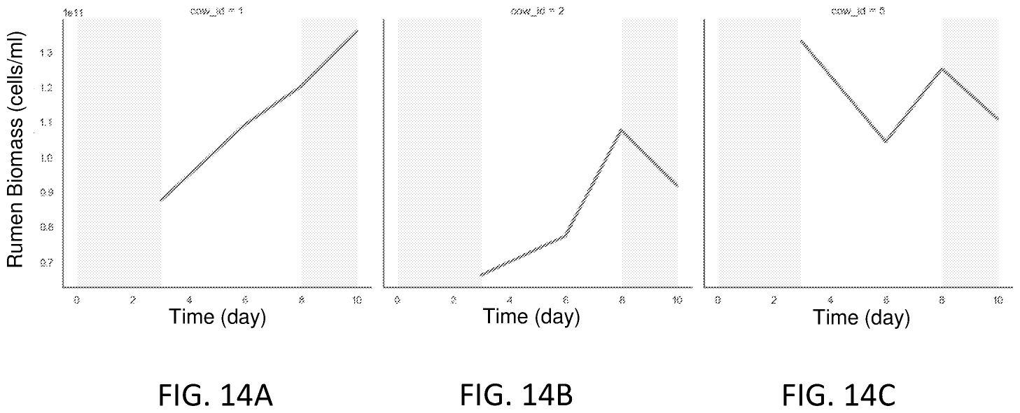

FIGS. 13A, 13B, and 13C display results associated with pH measurements obtained from the three cows in the study during the course of the study. As shown, samples from all three cows exhibited progressive decrease in pH with the induction of acidosis challenge. FIGS. 14A, 14B, and 14C display results associated with changes in rumen biomass obtained from the three cows in the study during the course of the study. As shown, samples from all three cows exhibited progressive increase in rumen biomass with the induction of acidosis challenge.

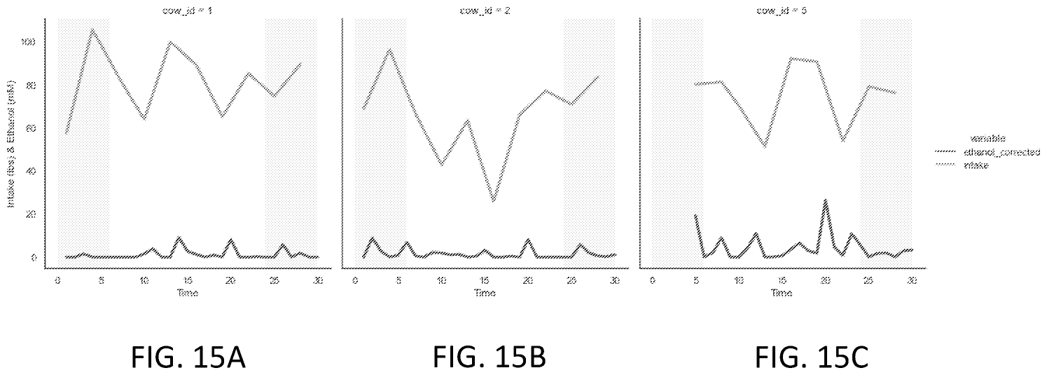

FIGS. 15A, 15B, and 15C display results associated with measurements of concentration of ethanol and measurements of intake obtained from the three cows in the study during the course of the study. As shown, concentration of ethanol increased to elevated levels in samples from all three cows with the induction of acidosis challenge. Notably, animal with ID 5 (results of which is shown in FIG. 14C), was found to have had limited access to feed before day 1 of the study, and was observed to be slug feeding on day 1. This animal showed clinical signs of bathypnea (deep breathing) at time point 20 after days of high intake.

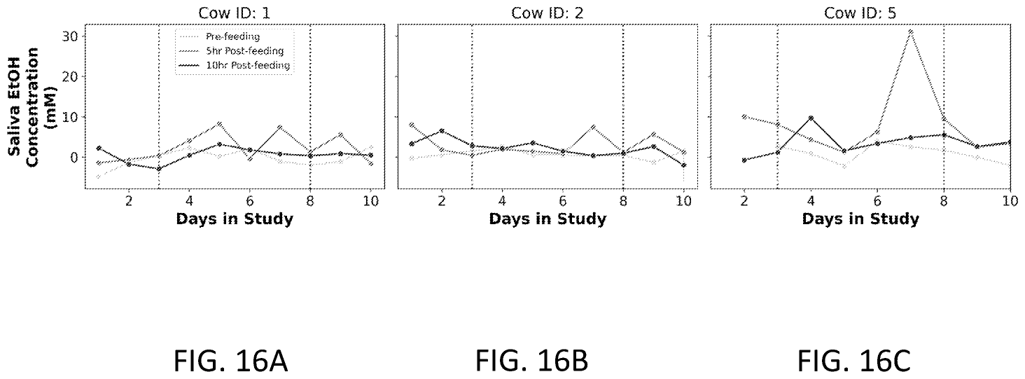

FIGS. 16A, 16B, and 16C display results associated with measurements of concentration of ethanol obtained from the three cows in the study during the course of the study, using systems and methods disclosed herein. As shown, concentration of ethanol increased to statistically greater amount compared to a predetermined baseline measurement (see FIG. 11B) in samples from all three cows with the induction of acidosis challenge. The three lines in each plot correspond to the three different sampling time per day, as indicated in the schedule in FIG. 12A.

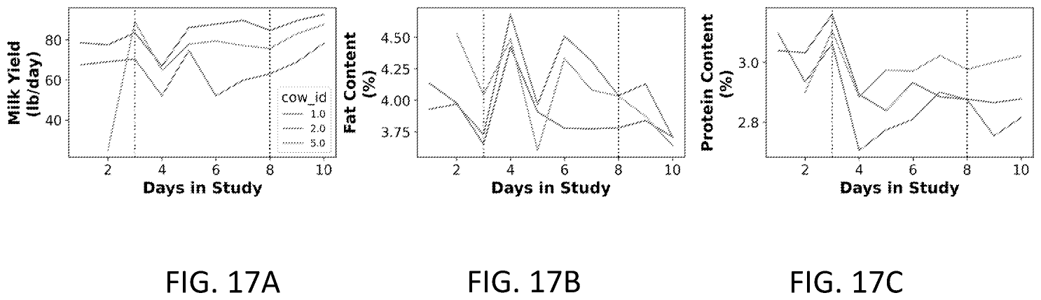

FIGS. 17A, 17B, and 17C display results associated with changes in milk production, fat content in the milk, and protein content in the milk, obtained from the three cows in the study during the course of the study, using systems and methods disclosed herein. As shown, diet changes induced acidosis in the lactating dairy cows, which is reflected by the reduced milk production and reduced components produced during the acidosis challenge (period between the dotted lines).

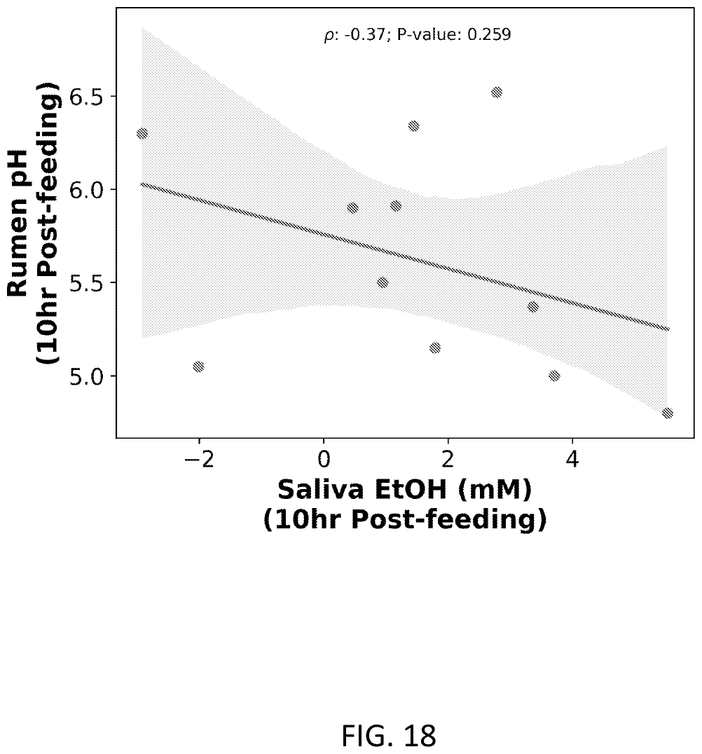

FIG. 18A is a plot showing a relationship between rumen pH and a concentration of ethanol in the saliva sample of the animal. As shown, there is a statistically significant negative correlational relationship between rumen pH and the measure concentration of ethanol in the saliva (correlation coefficient=−0.37, p-value 0.259). This result showed that measuring ethanol concentration in the saliva can be used as an accurate measure of a corresponding change in rumen pH that is associated with a state of acidosis in the animal.

### Experiment III: Solvent Removal

This experiment was designed to study a rate or efficiency of solvent removal using high porosity food grade components. The results from the study were used to generate indicators for an ADS described herein, to recommend potential carriers that can be used to define food supplements. Such food supplements can be recommended to bring an animal on a state or path to recovery from acidosis and/or pathophysiologies associated with acidosis.

The study included preparing a series of samples, each sample in the series containing a predetermined amount of ethanol. Five samples designated treatment samples also contained an additive in the form of a carrier configured to absorb ethanol. One sample that was designated control did not have any additive. Sample preparation included producing a 1 L volume of 0.5% Ethanol (w/w)-ethanol solution. Exactly 45 mLs of the ethanol solution was transferred in aliquots into 12 serum bottles. About 5.0 g of the following components were added into each bottle (in duplicates) in sequence—Control (no additive) bottles 1 and 2, Kaolin in bottles 3 and 4, Bentonite in bottles 5 and 6, Charcoal Powder in bottles 7 and 8, Bone Meal in bottles 9 and 10, and Charcoal Granules in bottles 11 and 12.

The contents of the bottles were mixed and then immediately 1 mL was removed from each bottle at a first timepoint (Timepoint “0 hr”). Serum was prepared in a serum bottle. The serum bottle was stoppered and incubated at 37-39° C. Following incubation, the 1 mL aliquot was centrifuged for more than 10,000×g for 5 minutes. The contents were then filtered, and the supernatant was extracted with a 0.22 μm sterile syringe and filtered into an HPLC vial. This was done for each treatment sample, and the vials were stored refrigerated at 4° C. until they were ready to analyze.

At 1 hr, 2 hr, and 5 hr timepoints, the contents of the bottles were mixed and immediately 1 mL was removed from each bottle at the corresponding timepoint (Timepoint “1 hr”, “2 hr” and “5 hr”), the serum bottle was stoppered and incubated at 37-39° C. Following incubation, the 1mL aliquot was centrifuged for more than 10,000×g for 5 minutes. The contents were then filtered, and the supernatant was extracted with a 0.22 μm sterile syringe and filtered into an HPLC vial. This was again done for each treatment sample in duplicates and at each time point, and the vials were stored refrigerated at 4° C. until they were ready to analyze.

For measuring a change in amount of ethanol, HPLC analysis was used. The procedure was as follows:
(a) Produce a 5-point standard curve for ethanol by creating solutions at 0.5%, 0.4%, 0.3%, 0.2% and 0.1% Ethanol (w/w). Sterile filter the solutions into HPLC vials, as described above.
(b) Load standards and samples into HPLC. Run samples through a 250 mM Agilent Hi-Plex H Column at 0.5 mLs/min, 10 minute run time, 35° C. column temperature, 35° C. RI detector with a 5 mM H₂SO₄ mobile phase. (parameters provided as an example, similar analysis can also be performed alternatively using, for example, a Bio-Rad Column Aminex HPX-87P #1250098 at a rate of 0.6 mL/min, at a column temperature of 85° C. Column Temp, 55° C. RI detector for 22-40 minutes with a water mobile phase.)
(c) Determine the peak area generated from the analysis of the standards and create a calibration curve.
(d) Calculate the concentration of each sample.

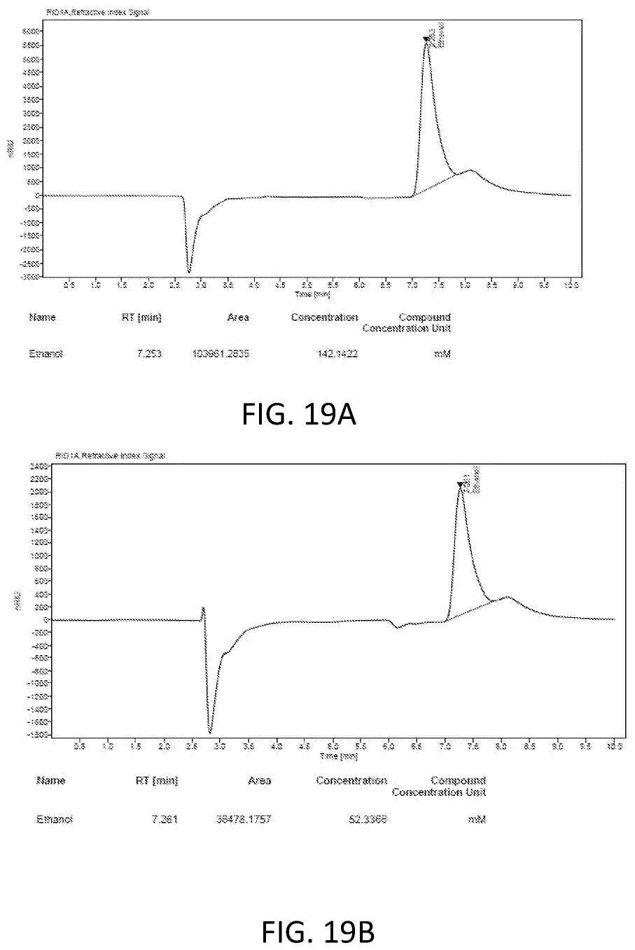

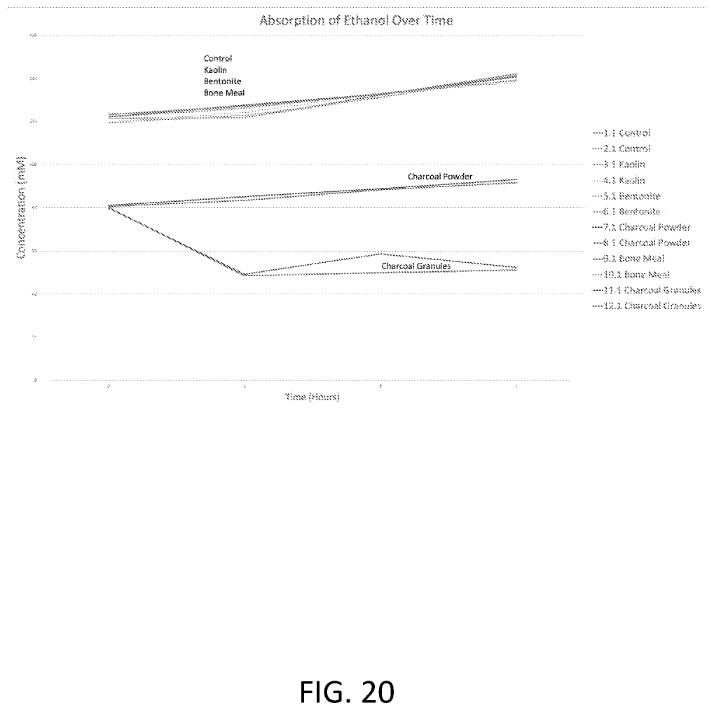

Examples of the curves generated are shown in FIG. 19A (control sample 1 at 5 hr. Timepoint) and 19B (sample with Charcoal granules 1 at 5 hr. Timepoint), and the summary of the results of the study are shown in the plot in FIG. 20. As shown in the summary in FIG. 20, Charcoal granules exhibited the best ability to absorb a quantity of ethanol. Charcoal powder was second best in the list tested, while the remaining did not deviate from the control sample significantly. Given the results, charcoal granules and charcoal powder appear to be top additives recommended as carriers for feed supplements to provide an animal diagnosed to have a state of acidosis, using systems and/or methods described herein.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which examples have been provided. The acts performed as part of the methods may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

Some embodiments and/or methods described herein can be performed by a different software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Python, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Lengthy table referenced here
US20250314672A1-20251009-T00001
Please refer to the end of the specification for access instructions.

Lengthy table referenced here
US20250314672A1-20251009-T00002
Please refer to the end of the specification for access instructions.

Lengthy table referenced here
US20250314672A1-20251009-T00003
Please refer to the end of the specification for access instructions.

Lengthy table referenced here
US20250314672A1-20251009-T00004
Please refer to the end of the specification for access instructions.

Lengthy table referenced here
US20250314672A1-20251009-T00005
Please refer to the end of the specification for access instructions.

Lengthy table referenced here
US20250314672A1-20251009-T00006
Please refer to the end of the specification for access instructions.

### INCORPORATION BY REFERENCE

All references, articles, publications, patents, patent publications, and patent applications cited herein are incorporated by reference in their entireties for all purposes, including PCT Application Nos. PCT/US2017/012573, PCT/US2020/020311, and PCT/US2021/025264.

However, mention of any reference, article, publication, patent, patent publication, and patent application cited herein is not, and should not be taken as, an acknowledgment or any form of suggestion that they constitute valid prior art or form part of the common general knowledge in any country in the world.

LENGTHY TABLES
The patent application contains a lengthy table section. A copy of the table is available in electronic form from the USPTO web site (https://seqdata.uspto.gov/?pageRequest=docDetail&DocID=US20250314672A1). An electronic copy of the table will also be available from the USPTO upon request and payment of the fee set forth in 37 CFR 1.19(b)(3).

1. A method of diagnosing a state of health of an animal, comprising:
receiving a sample of a bodily fluid or gas from an oral or nasal cavity of the animal;
measuring a quantity of at least one of an identified solvent or an identified gas in the sample; and
determining a state of health associated with the animal based on the quantity of the identified solvent.

2. The method of claim 1, wherein the identified solvent is at least one of an alcohol, acetone, succinate, or an acetaldehyde.

3. The method of claim 1, wherein the identified solvent is at least one of ethanol, isopropanol, acetone, or butanol.

4. The method of claim 1, wherein the identified gas is carbon dioxide

5. The method of claim 1, wherein the animal is a ruminant, and the sample is a sample of bodily fluid, and the bodily fluid is saliva.

6. The method of claim 1, further comprising:
comparing the quantity of the at least one of an identified solvent or an identified gas with a predetermined baseline value; and
determining the state of health associated with the animal to include a state of acidosis based on the quantity of the at least one of an identified solvent or an identified gas being greater than the predetermined value.

7. The method of claim 1, further comprising:
comparing the quantity of the at least one of an identified solvent or an identified gas with a predetermined value; and
determining the state of health associated with the animal to not include a state of acidosis based on the quantity of the at least one of an identified solvent or an identified gas being greater than the predetermined value.

8. The method of claim 6, further comprising:
preparing a feed supplement including at least one of (i) a synthetic microbial ensemble including one or more identified microorganism strains, or (ii) a carrier configured to adsorb a solvent; and
combining feed supplement with feed to provide the animal, the feed supplement configured to induce a recovery from the state of acidosis.

9. The method of claim 8, wherein the carrier is at least one of Zeolite, charcoal, activated granulated charcoal, bone ash, bone charcoal, diatomaceous earth, kaolin, and clay.

10. The method of claim 6, further comprising:
preparing a feed supplement including a microbial growth inhibitor configured to inhibit solventogenesis; and
combining feed supplement with feed to provide the animal, the feed supplement configured to induce a recovery from the state of acidosis.

11. The method of claim 10, wherein the microbial growth inhibitor is at least one of ascorbic acid, benzoic acid, propionic acid, sodium sulfite, sulfur dioxide, sodium nitrite, and fumaric acid.

12. A method of providing a feed supplement to an animal comprising:
identifying at least one of a synthetic microbial ensemble including one or more identified microorganism strains, or a carrier configured to adsorb a solvent; and
generating a feed supplement by combining an amount of the synthetic microbial ensemble or an amount of the carrier with feed, the feed supplement configured to be provided to the animal.

13. The method of claim 12, further comprising:
receiving an indication that the animal is associated with a state of acidosis; and
determining the amount of the carrier based on the indication, the feed supplement configured to induce a recovery from the state of acidosis.

14. The method of claim 13, wherein the indication is a first indication, the amount is a first amount and the feed supplement is a first feed supplement, the method further comprising:
receiving a second indication that the animal is associated with a recovery from the state of acidosis; and
determining a second amount of the carrier based on the second indication, the feed supplement configured to induce a maintenance of the recovery from the state of acidosis.

15. The method of claim 12, wherein the carrier is at least one of Zeolite, charcoal, activated granulated charcoal, bone ash, bone charcoal, diatomaceous earth, kaolin, and clay.

16. The method of claim 12, wherein the solvent is ethanol.

17. The method of claim 12, wherein the identifying the carrier is based on absorptive properties of the carrier for ethanol.

18. A method of providing a feed supplement to an animal comprising:
identifying a microbial growth inhibitor configured to inhibit solventogenesis; and
generating a feed supplement by combining an amount of the microbial growth inhibitor with feed to provide an animal, the feed supplement configured to induce in the animal a change in a health status.

19. The method of claim 18, wherein the health status is a state of acidosis, the change in health status being a recovery from the state of acidosis.

20. The method of claim 18, wherein the microbial growth inhibitor is at least one of ascorbic acid, benzoic acid, propionic acid, sodium sulfite, sulfur dioxide, sodium nitrite, and fumaric acid.

21. The method of claim 18, wherein the microbial growth inhibitor is at least one of ascorbic acid, benzoic acid, propionic acid, sodium sulfite, sulfur dioxide, sodium nitrite, and fumaric acid.

22. An apparatus, comprising:
a sampling structure configured to capture a sample, the sample being at least one of a bodily fluid or a gas from an oral or nasal cavity of an animal; and
a chamber operatively coupled to the sampling structure to access at least a portion of the sample, the chamber configured to process the sample to quantify an amount of at least one of a solvent or gas in the sample.

23. The apparatus of claim 22, wherein the sampling structure includes a test strip of material configured to detect an amount of the at least one of a solvent or gas in the sample.

24. The apparatus of claim 23, wherein the material is configured to detect the amount of the at least one of a solvent or gas in the sample based on at least one of a peroxidase based assay or a colorimetric assay.

25. The apparatus of claim 23, wherein the material is configured to provide a visible indication based on a detection of an amount of a solvent that is within a predetermined range of concentrations.

26. The apparatus of claim 25, wherein the solvent is ethanol and the predetermined range of concentrations is 0 mM-10 mM.

27. A non-transitory processor-readable medium storing code representing instructions to be executed by a processor, the instructions comprising code to cause the processor to:
obtain a measurement of a property associated with a solvent in a sample of bodily fluid from an animal;
compare the measurement of the property associated with the solvent against a predetermined baseline measurement of the property associated with the solvent; and
determine (i) a state of acidosis based on the measurement being greater than the predetermined baseline measurement, or (ii) a state of no acidosis based on the measurement being lesser than the predetermined baseline measurement.

28. The non-transitory processor-readable medium of claim 27, further comprising code to cause the process to:
generate a feed recommendation in response to the determination of (i) the state of acidosis, or (ii) the state of no acidosis.

29. The non-transitory processor-readable medium of claim 28, wherein the feed recommendation includes providing the animal a feed supplement including at least one of (i) a synthetic microbial ensemble including one or more identified microorganism strains, or (ii) a carrier configured to absorb a solvent, the feed supplement configured to induce a recovery from the state of acidosis.

30. The non-transitory processor-readable medium of claim 29, wherein the carrier is at least one of Zeolite, charcoal, activated granulated charcoal, bone ash, bone charcoal, diatomaceous earth, kaolin, and clay.

31. The non-transitory processor-readable medium of claim 27, further comprising code to cause the processor to:
predict, in response to the determination of the state of acidosis, a prediction of progression of the state of acidosis.

32. The non-transitory processor-readable medium of claim 27, further comprising code to cause the processor to:
predict, in response to the determination of the state of acidosis and an indication of providing the feed supplement, a prediction of progression of recovery from the state of acidosis.
